(19) United States
(12) Patent Application Publication
(10) Pub. No.: US 2007/0202138 A1
(43) Pub. Date: Aug. 30, 2007

(54) ANTIMICROBIAL COMPOSITIONS AND METHOD

(75) Inventor: Lawrence A. Funt, Bethesda, MD (US)

Correspondence Address: PEARNE & GORDON LLP, 1801 EAST 9TH STREET, SUITE 1200, CLEVELAND, OH, 44114-3108, US

(73) Assignee: BIOFILM INNOVATIONS GROUP, LLC, Bethesda, MD (US)

(21) Appl. No.: 11/680,143

(22) Filed: Feb. 28, 2007

Related U.S. Application Data
(60) Provisional application No. 60/777,385, filed on Feb. 28, 2006.

(51) Int. Cl. A61K 31/205; A61L 2/18

(52) U.S. Cl. 424/405; 422/28; 424/400; 514/150; 514/554

(57) ABSTRACT

Compositions for antimicrobial, antibacterial, antiviral, fungicidal and sporicidal applications comprise a mixture of alkyl betaine and alkyl amine oxide components together with a protonating agent. The compositions are particularly effective in the treatment and elimination of microorganisms in planktonic cell form as well as in sessile cell form in biofilms. The compositions may be applied in the form of sprays and foams as well as in liquid forms, as a solution or as a balm, as the sole active ingredient or with other active ingredients together with carriers or diluents.

This application claims the priority of U.S. Provisional Application No. 60/777,385, filed Feb. 28, 2006.

### BACKGROUND OF THE INVENTION AND RELATED ART

The present invention relates to compositions and methods of using the compositions for antimicrobial, antibacterial, antiviral, fungicidal and sporicidal applications. The compositions and methods are particularly effective in the treatment and elimination of microorganisms in planktonic cell form as well as in sessile cell form in biofilms. The compositions and methods are useful in the treatment of humans and animals as well as inanimate objects, devices and facilities as an antimicrobial, sterilant and/or disinfectant.

Medical instruments are typically sterilized or disinfected by introducing them into high temperature and high-pressure autoclaves. Although effective in killing microorganisms, the autoclaves have several significant disadvantages. Autoclaves are typically expensive and have high maintenance costs due to the operating conditions. The extreme pressure and temperature conditions in autoclaves preclude their use in connection with many medical instruments that are sensitive to such extreme environments. Further, autoclaves typically require long cycle periods which range from several minutes to several hours, or even days.

The use of ethylene oxide gas in sealed sterilization chambers at elevated pressures has provided an alternative to autoclaves. These techniques are characterized by long cycle times requiring long exposure times in vacuum and subsequent aeration cycles. Moreover, ethylene oxide is not effective in respect to many medical devices, and it is extremely toxic.

The problems of sterilization become substantially more difficult when biofilms are encountered. Biofilms provide a protective environment for microorganisms existing therein. The organization, protective mechanisms, and cooperation of the various species residing within the biofilms are recognized. Dental plaque, a common biofilm, has been found to contain more than 500 types of microorganisms including bacteria, fungi, viral, spores and even amoebas.

Biofilms are ubiquitous. They are found in a wide range of animal and plant environments as well as inanimate environments such as medical equipment and apparatus, especially where liquids are available to provide a source of nutrients. In all cases, the extra-cellular matrix of the biofilm secures the microorganisms together and to a recipient surface. The matrix also serves to provide protection since a substance will have a difficult time diffusing into the center of the matrix if it reacts with the cells or the matrix material it encounters along the way. In turn, environmental changes result within the matrix and a variety of chemical environments arise with corresponding differences in the cells, even though they are genetically identical, there are changes in genetic expression and phenotypic changes.

### SUMMARY OF THE INVENTION

Applicant has now discovered that selected compositions are effective in the control and elimination of microorganisms in planktonic cell form as well as in sessile cell form in biofilms. The compositions are effective for antimicrobial, antibacterial, antiviral, fungicidal and sporicidal treatments. The compositions are substantially nontoxic and otherwise do not harm or damage animal tissue or cells. The compositions are particularly useful as sterilants/disinfectants at room temperature and with relatively short treatment times and dilute concentrations.

Sterilization is defined as the complete killing of all foreign organisms. Herein, sterilization is deemed to be indicated by the inactivation (or killing) of a significant challenge (e.g., one million cfu) of Bacillus stearothermophilus spores at ambient or room temperature conditions, upon contact with an effective amount of the compositions of the present invention. If a process is successful in inactivating a significant challenge of B. stearothermophilus spores, then it is recognized that all pathogenic bacterial spores, as well as viruses, fungi, and vegetative bacteria exposed to those conditions at that time, are also inactivated. Editor, Joseph M. Ascenszi, Handbook of Disinfectants and Antiseptics, Marcel Dekker, Inc., 1996.

Disinfection is understood to be the selective elimination of selected undesirable microorganisms to prevent their transmission, i.e., the reduction of the number of infectious organisms to a value below that necessary to cause infection. Antisepsis is the application of an antimicrobial to skin or other living tissue to inhibit the growth of and/or destroy microorganisms.

The effectiveness of the compositions in the control and killing of biofilms is most surprising. It is believed that the compositions themselves disrupt the physical state of the biofilm to gain better access to the sessile cells therein and enhance the antimicrobial, antibacterial, antiviral, fungicidal and sporicidal effects of the compositions per se on the sessile cells after adoption of the biofilm phenotype.

The active ingredients or components of the compositions comprise a mixture of alkyl betaine and alkyl amine oxide components together with a protonating agent. The mixture may be formed by combining the betaine and amine oxide components, and than adding the protonating agent or acid together with a suitable solvent to provide the overall resulting mixture. The concentration of the active betaine and amine oxide ingredients may range from about 0.01 part to about 40 parts by weight per 100 parts total, and, more preferably, from about 0.02 part to about 20 parts. As used herein, the reference to “part” or “parts” is by weight based on 100 parts total of the mixture of the composition discussed unless otherwise indicated by the context.

The effective ingredients of the inventive compositions comprise in admixture:
(a) a mixture of two alkyl-N-betaines,
(b) an alkyl-N,N-dimethylamine oxide, and
(c) a protonating agent, such as hydrochloric acid, acetic acid or citric acid, in an amount sufficient to adjust the pH of the overall composition in the range of from about 4 to about 7.5.

### Each of the betaines and the amine oxide is present in an amount ranging from about 0.01 part to about 20 parts, and more preferably, from about 0.02 part to about 10 parts.

The betaine compositions are:

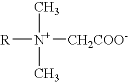

where R is a mixture of higher alkyl having from 12 to 14 carbon atoms. Illustrative of such mixtures are lauryl-N-betaine and myristyl-N-betaine in lauryl/myristyl mixture ratios of from about 30:70 to about 70:30. More preferably, the mixture ratio is from about 60:40 to about 50:50.
The amine composition is:

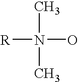

where R is a higher alkyl containing 16 carbon atoms, in the form of a cetyl radical. Accordingly, the amine composition comprises cetyl-N,N-dimethylamine oxide.

The use of a protonating agent supplies the required pH and cooperates in the effectiveness of the compositions and processes herein. Illustrative protonating agents include any suitable organic or inorganic acid, such as hydrochloric acid, phosphoric acid, sulfuric acid, citric acid, acetic acid, nicotinic acid, and the like. The solution may have a pH in the range of from about 4 to about 7.5, and more preferably, from about 4 to about 5, and most preferably, about 4.85. The protonating agent is contained in a suitable solvent such as water or a suitable lower alcohol, C1 to C4 aliphatic alcohol, or combinations thereof. With the use of buffers, effective kill is achieved at pH values in the range of from about 6 to about 7.4. The pH may be lowered with the use of HCl and increased with the use of phosphate buffered saline.

In accordance with the invention, the compositions and methods have utility in sterilant applications at room temperature and atmospheric pressure, and also at elevated temperatures and pressures, with direct application of the sterilant to the article to be sterilized. The compositions and methods are particularly useful in health care facilities as well as field environments. Similarly, the compositions and methods may be used in industrial applications, especially those involving water supply or processing.

The compositions are useful as sterilants for application to medical implements, especially as may be encountered in military uses under field conditions. In this respect, the sterilant also acts as a cleaner or disinfectant, or a component thereof. The compositions are safe for application to human tissue and for human ingestion.

The compositions have antimicrobial properties including a high-level antimicrobial kill of fungi, gram positive and gram negative bacteria and spore forming microbes. Therapeutic and prophylactic effectiveness has been confirmed in connection with a variety of activities described hereinafter. And, as noted above, the compositions are effective against planktonic cell and sessile cell forms as well as a biofilm combatant including penetration, dislodgement and/or disintegration of the biofilm structure.

The compositions of the present invention are surfactant in nature including hydrophobic molecule ends. The betaines are recognized as amphoteric surfactants. The surfactant characteristics also cause the compositions to display a tendency to foam in the air when mixed in a liquid dispensing action such as discharge from a pump container. The resulting foam will be maintained for less than about a minute under ambient conditions, room temperature and atomospheric pressure. Thereafter, the foam collapses to form a continuous film. The film has a tendency to be retained on the supporting substrate such as inorganic metal or glass surfaces and organic surfaces such as human skin. The composition is therefore useful to form a “liquid bandage”. The resulting film provides prophylactic-type protection in the nature of a barrier as well as antimicrobial, antibacterial, antiviral, fungicidal and sporicidal effects.

The inventive compositions are also useful in connection with devices requiring a relatively contamination free or disinfected or sterile environment for frictionally engaged moving parts. In such an environment, the compositions have been found to act as a lubricant as well as a sterilant/disinfectant. For example, medical instruments or dental instruments such as dental hand pieces.

### BRIEF DESCRIPTION OF THE DRAWINGS

FIG. 1 is a graph showing Pseudomonas aeruginosa kill rate over time following treatment with the compositions of the invention and following treatment with several comparative compositions;

FIG. 2 is a graph showing Candida albicans kill rate over time following treatment with the compositions of the invention and following treatment with several comparative compositions;

FIG. 3 is a graph showing E. coli kill rate over time following treatment with the compositions of the invention and following treatment with several comparative compositions;

FIG. 4 is a graph showing Bacillus stearothermophilus kill rate over time following treatment with the compositions of the invention and following treatment with several comparative compositions;

FIG. 5 is a graph showing Candida albicans kill rate over time following treatment with the compositions of the invention at reduced concentrations;

FIG. 6 is a graph showing E. coli kill rate over time following treatment with the compositions of the invention at reduced concentrations;

FIG. 7 is a graph showing Pseudomonas aeruginosa kill rate over time following treatment with the compositions of the invention at reduced concentrations;

FIG. 8 is a graph showing Bacillus stearothermophilus kill rate over time following treatment with the compositions of the invention at reduced concentrations;

FIG. 9 is a graph showing mixed oral flora kill rate over time following treatment with the compositions of the invention at reduced concentrations;

FIG. 10 is a graph showing Methicillin-Resistant Staphylococcus aureus (MRSA) kill rate over time following treatment with the compositions of the invention;

FIG. 11 is a graph showing Staphylococcus aureus kill rate over time following treatment with the compositions of the invention;

FIG. 12 is a graph showing Acinetobacter baumannii kill rate over time following treatment with the compositions of the invention;

FIG. 13 is a graph showing Vancomycin-resistant Enterococci (VRE) kill rate over time following treatment with the compositions of the invention;

FIG. 14 is a graph showing the kill rates of Streptococcus pyogenes versus the compositions of the invention and various commercially available disinfectants;

FIGS. 15 through 26 are photomicrographs showing various biofilms treated with the compositions of the invention;

FIG. 27 is a graph reporting a survey count of the microbes, molds and Beta hemolytic pathogens present in untreated areas of a dental facility;

FIG. 28 is a graph similar to FIG. 28 reporting the count of the microbes, molds and Beta hemolytic pathogens after five minutes following treatment with the composition of the invention; and

FIG. 29 is a graph similar to FIG. 29 reporting the count of the microbes, molds and Beta hemolytic pathogens after one minute following a spray treatment with the composition of the invention.

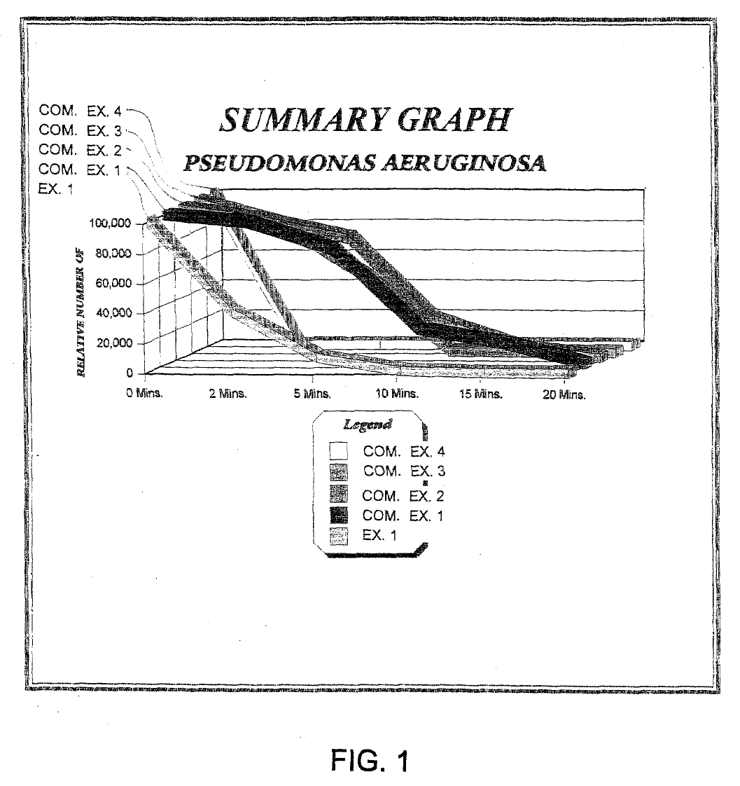

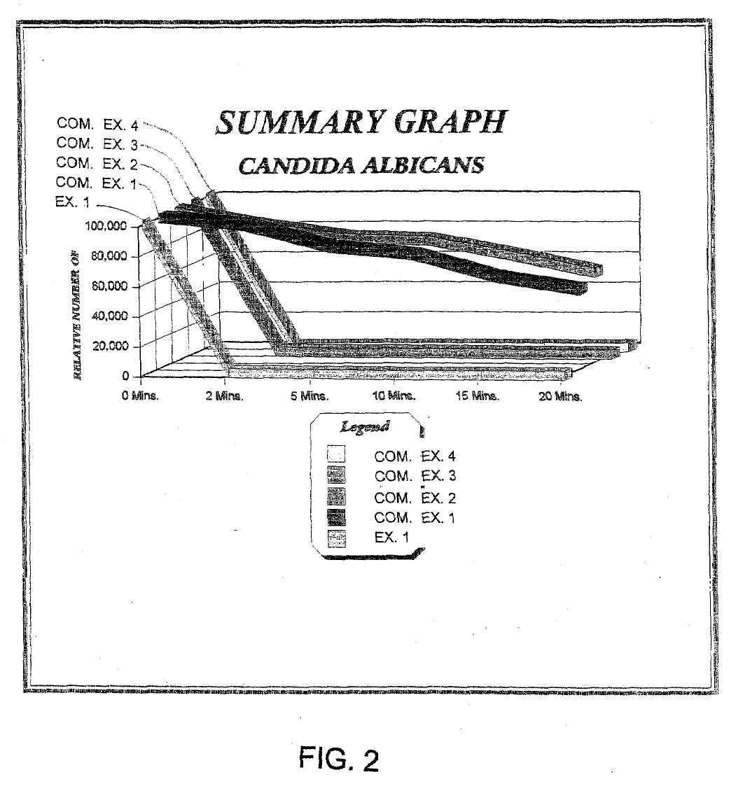

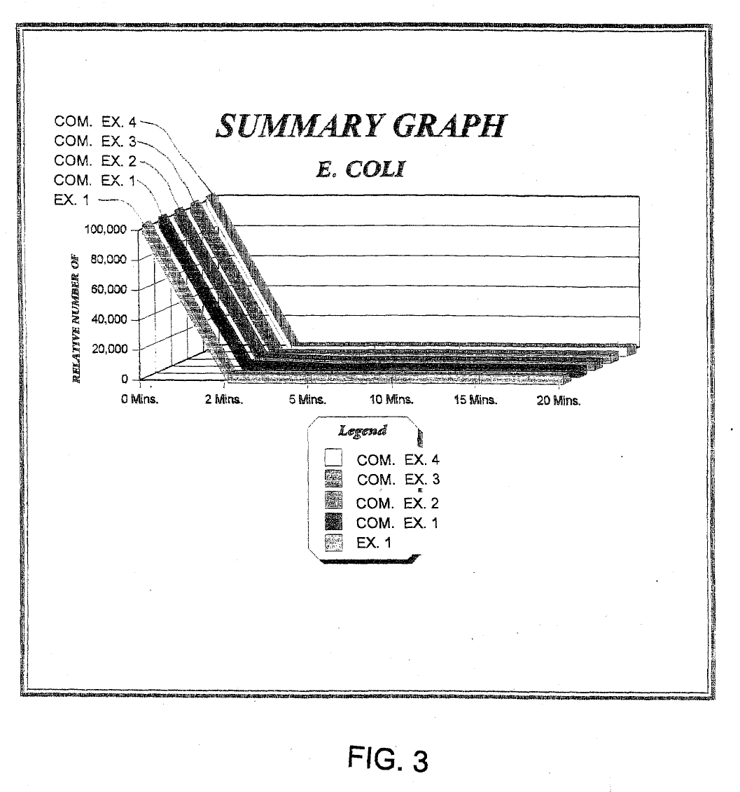

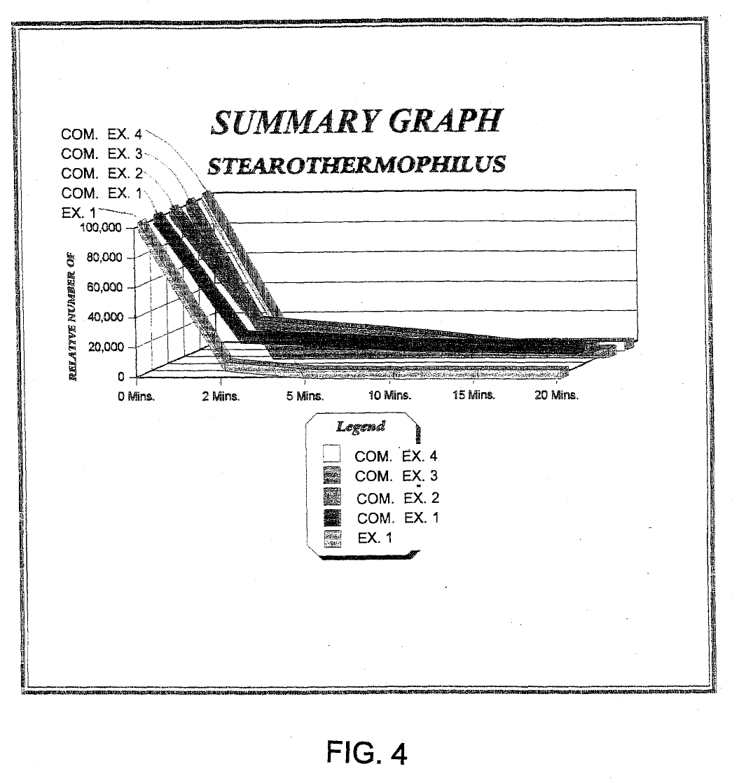

### DETAILED DESCRIPTION OF THE INVENTION

The compositions may be applied or administered in conventional manners in aerosol or foam forms as well as in liquid form, as a solution or as a balm, as the sole active ingredient or with other active ingredients together with carriers or diluents as are known in the art. The compositions and methods of the present invention are initially described herein with respect to their sterilant applications and sterilizing utilities.

In the following examples and comparative examples, the components are reported in weight percent based on the total weight or the numerically corresponding parts per 100 parts total, unless otherwise indicated by the text or context of the discussion. Distilled water is used as the solvent in all of the examples to form the overall mixture or to prepare dilutions thereof.

### Example 1

The ingredients of the composition of Example 1 comprise in admixture:
(a) Lauryldimethylbetaine, 0.84% by weight,
(b) Myristyldimethylbetaine, 0.36% by weight,
(c) Cetyldimethyl amine oxide, 1.20% by weight, and
(d) citric acid in an amount sufficient to adjust the pH of the overall composition to about 4.85.

The betaine and amine oxide active ingredients of the composition may be combined at room temperature with mixing. The acid may be combined with the foregoing ingredients or subsequently combined together with distilled water.

### Comparative Example 1

Comparative Example 1 is prepared using the same procedures as described above and comprises in admixture:
(a) Cetyldimethylbetaine, 1.2% by weight,
(b) Myristyldimethyl amine oxide, 1.2% by weight,
(c) citric acid in an amount sufficient to adjust the pH of the overall composition to about 4.85.

### Comparative Example 2

Comparative Example 2 is prepared using the same procedures as described above and comprises in admixture:
(a) Lauryldimethylbetaine 1.26%,
(b) Myristyldimethylbetaine 0.54%,
(c) Cocoamidopropyl amine oxide 1.80%, and
(d) citric acid in an amount sufficient to adjust the pH of the overall composition to about 4.85.

### Comparative Example 3

Comparative Example 3 is prepared using the same procedures as described above and comprises in admixture:
(a) Lauryldimethylbetaine 1.26%,
(b) Myristyldimethylbetaine 0.54%,
(c) Myristyl-bis (2-hydroxyethyl) amine oxide 1.26%,
(d) Cetyl-bis (2-hydroxyethyl) amine oxide 0.54%, and
(d) citric acid in an amount sufficient to adjust the pH of the overall composition to about 4.85.

### Comparative Example 4

Comparative Example 4 is prepared using the same procedures as described above and comprises in admixture:
(a) Cetyldimethylbetaine 1.21%,
(b) Lauryldimethylbetaine 0.85%,
(c) Myristyldimethylbetaine, 0.36%,
(d) Myristyldimethyl amine oxide, 1.70%,
(d) Cetyldimethyl amine oxide 0.73%, and
(e) citric acid in an amount sufficient to adjust the pH of the overall composition to about 4.85.

### Comparative Example 5

Comparative Example 5 is prepared using the same procedures as described above and comprises in admixture:
(a) Lauryldimethylbetaine 2.43%,
(b) Cetyldimethyl amine oxide 2.94%,
(c) citric acid in an amount sufficient to adjust the pH of the overall composition to about 4.85.

### Comparative Example 6

Comparative Example 6 is prepared using the same procedures as described above and comprises in admixture:
(a) Myristyldimethylbetaine 2.43%,
(b) Cetyldimethyl amine oxide 2.94%,
(c) citric acid in an amount sufficient to adjust the pH of the overall composition to about 4.85.

### Comparative Example 7

Comparative Example 7 is prepared using the same procedures as described above and comprises in admixture:
(a) Cocodimethylbetaine 2.49%,
(b) Cocodimethyl amine oxide 2.49%,
(c) citric acid in an amount sufficient to adjust the pH of the overall composition to about 4.85.

### The Cocodimethylbetaine is a commercially available blend of alkyl substituted betaines with the following approximate compositions of alkyl components by weight percent.

C₈-3.2%

C₁₀-6.3%

C₁₂-51.9%

C₁₄-20.7%

C₁₆-12.1%

C₁₈-5.9%

### The “coco” species is economically favored in many other applications, but not found particularly useful herein.

### Comparative Example 8

Comparative Example 8 is prepared using the same procedures as described above and comprises in admixture:
(a) Cocodimethylbetaine 2.49%,
(b) Cetyldimethyl amine oxide 2.49%,
(C) citric acid in an amount sufficient to adjust the pH of the overall composition to about 4.85.

### The “coco” species is again used in this comparative example.

The kill rates of the compositions Example 1 and Comparative Examples 1-4 were determined with respect to Pseudomonas aeruginosa, Candida albicans, E. coli, and Bacillus stearothermophilus. The kill rate of each of the compositions was determined by combining a 200 microliter dilution of the composition being tested with a 2 ml sample of bacteria containing two billion colony forming units (cfu's). It should be appreciated that conventional testing may be against several million cfu's. The mixture was maintained at 70° F., and 0.5 ml aliquots were withdrawn at various time points. The aliquots were plated-out using standard plate count methodology to determine the reduction of cfu's per time point.

The kill rates of the compositions of Comparative Examples 5-8 were determined with respect to E. coli using the above procedures and an initial bacteria sample containing one billion colony forming units.

TABLE 1

| | % | PSEUDOMONAS | CANDIDA | | BACILLUS |
|---|---|---|---|---|---|
| EXAMPLE | ACTIVE | AERUGINOSA | ALBICANS | E. COLI | STEAROTHERMOPHILUS |
| NO. | INGRED. | MINUTES TO COMPLETE KILL | | | |
| Ex. 1 | 2.40 | 5 | 2 | 2 | 2 |
| Com. Ex. 1 | 2.40 | 20 | Fail | 2 | 20+ |
| Com. Ex. 2 | 3.60 | 20 | Fail | 2 | 20+ |
| Com. Ex. 3 | 3.60 | 12 | 2 | 2 | 3 |
| Com. Ex. 4 | 4.85 | 2 | 2 | 2 | 2 |
| Com. Ex. 5 | 5.37 | | | 15 | |
| Com. Ex. 6 | 5.37 | | | 10 | |
| Com. Ex. 7 | 4.98 | | | 10 | |
| Com. Ex. 8 | 4.98 | | | 10 | |

As indicated, the composition of Example 1 effectively 14 kills the indicated organisms during relatively short exposure or contact times in the order of seconds or minutes. Moreover, the results are achieved at a lower concentration of active ingredients as compared with the compositions of Comparative Examples 3 and 4. The efficacy of the composition of Example 1 in the “kill” and limitation of growth of a panel of bacteria shows Example 1 to be a broad spectrum efficient anti-microbial agent.

### Example 2

The ingredients of the composition of Example 2 comprise in admixture:
(a) Lauryldimethylbetaine, 1.95 parts by weight,
(b) Myristyldimethylbetaine, 1.05 parts by weight,
(c) Cetyldimethyl amine oxide, 3.00 parts by weight, and
(d) citric acid in an amount sufficient to adjust the pH of the overall composition to about 4.85.

The betaine and amine oxide active ingredients of the composition may be combined at room temperature with mixing. The acid may be combined with the foregoing ingredients or subsequently combined together with distilled water. The resulting composition contains 5.00% active ingredients based on the weight of the betaine and amine oxide components, and for purposes herein, it is considered to be a 1:5 dilution used to make further dilutions as reported below.

### Example 3

The composition of Example 2 was further diluted using distilled water to provide a 1:10 dilution to prepare Example 3. Example 3 has a concentration of active ingredients, the total betaine and amine oxide components, equal to 2.50% by weight.

### Example 4

The composition of Example 2 was further diluted using distilled water to provide a 1:60 dilution ratio to prepare Example 4. Example 4 has a concentration of active ingredients, the total betaine and amine oxide components, equal to 0.41% by weight.

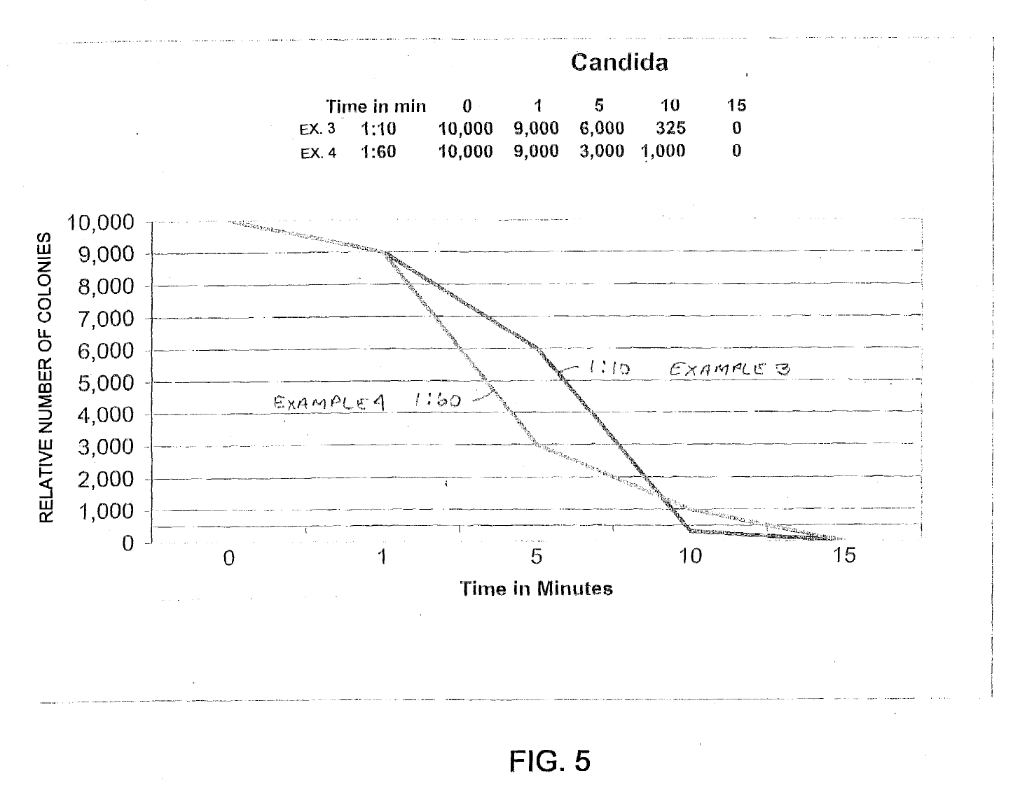

The effectiveness of kill of Examples 3 and 4 were measured against Candida albicans at room temperature beginning with an initial microbe count of one billion. The results are shown in FIG. 5. The initial kill rate over the first minute was similar for Examples 3 and 4. Thereafter, the more concentrated solution of Example 3 exceeded Example 4. However, both concentrations provided substantially 100% kill by 15 minutes.

These examples demonstrate that the compositions of the present invention are exceptionally effective against Candida albicans, one of the most difficult microbes to kill. Candida albicans is a member of the fungal family, primarily a yeast, but a dimorphic microbe, capable of developing a mold-like appearance under proper environmental conditions.

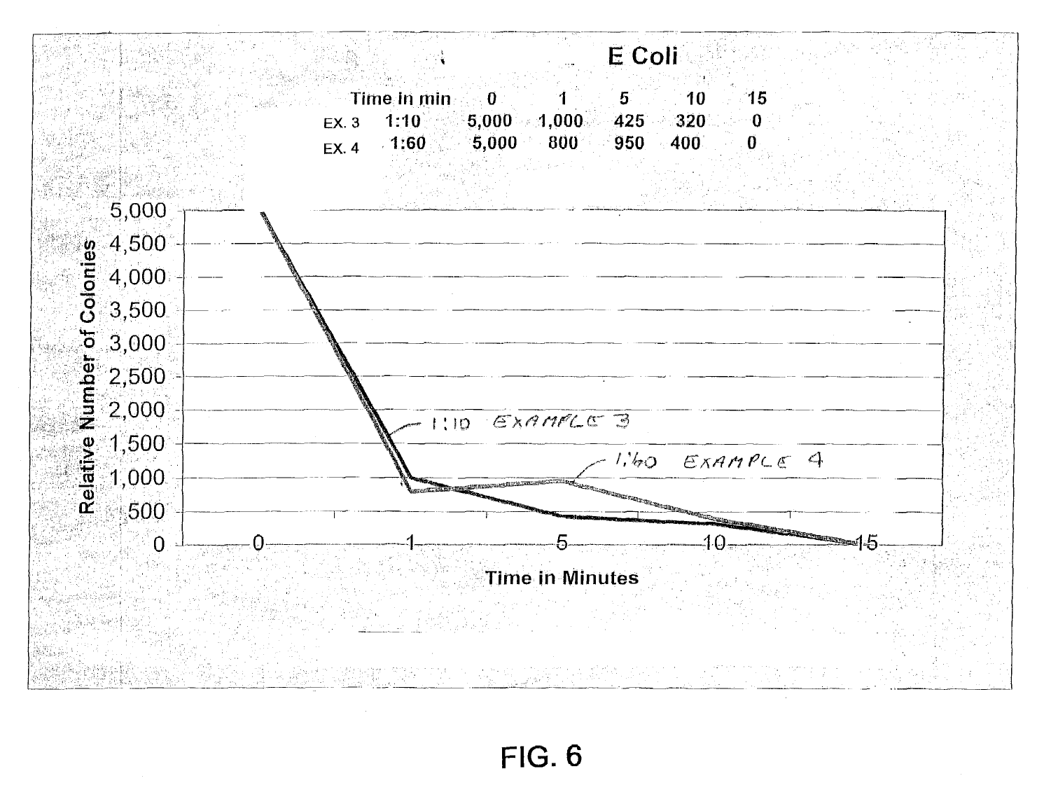

Referring to FIG. 6, the kill rate of Examples 3 and 4 against E. coli is reported. Again, at time zero, there were about one billion microbes present, and equivalence of kill is seen with substantially 100% kill being achieved after 15 minutes at room temperature.

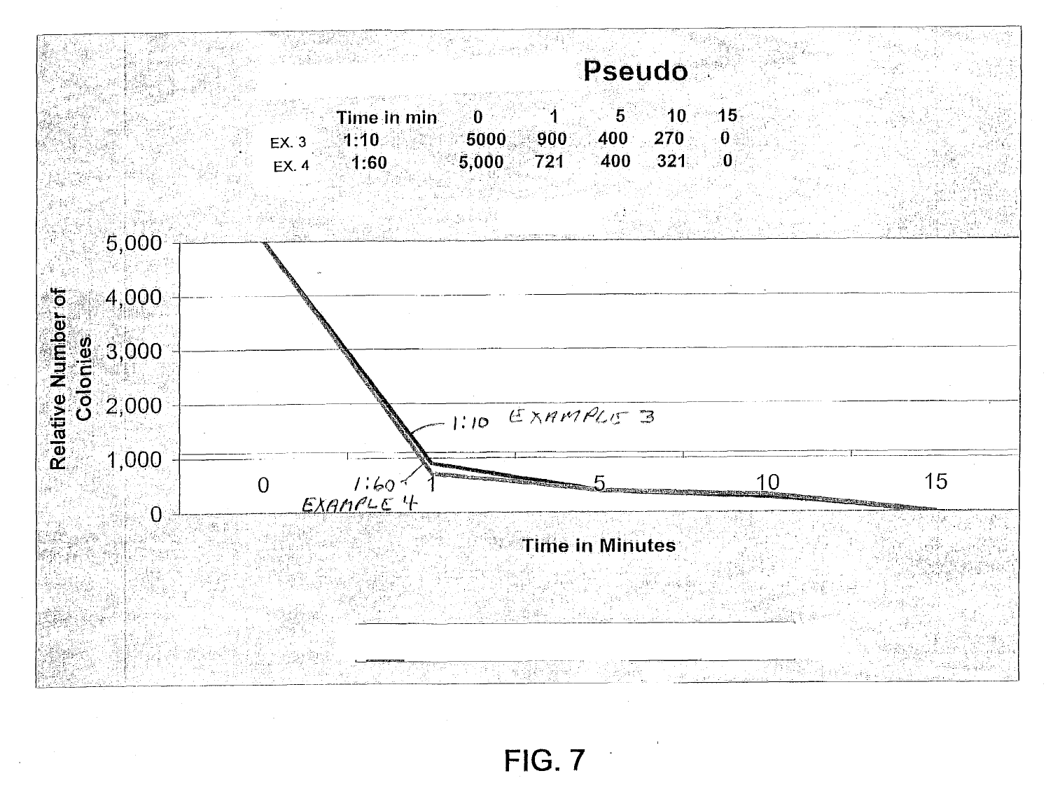

The kill rates of the compositions of Examples 3 and 4 against Pseudomonas aeruginosa are reported in FIG. 7. Again, equivalence of kill is seen with total kill by 15 minutes.

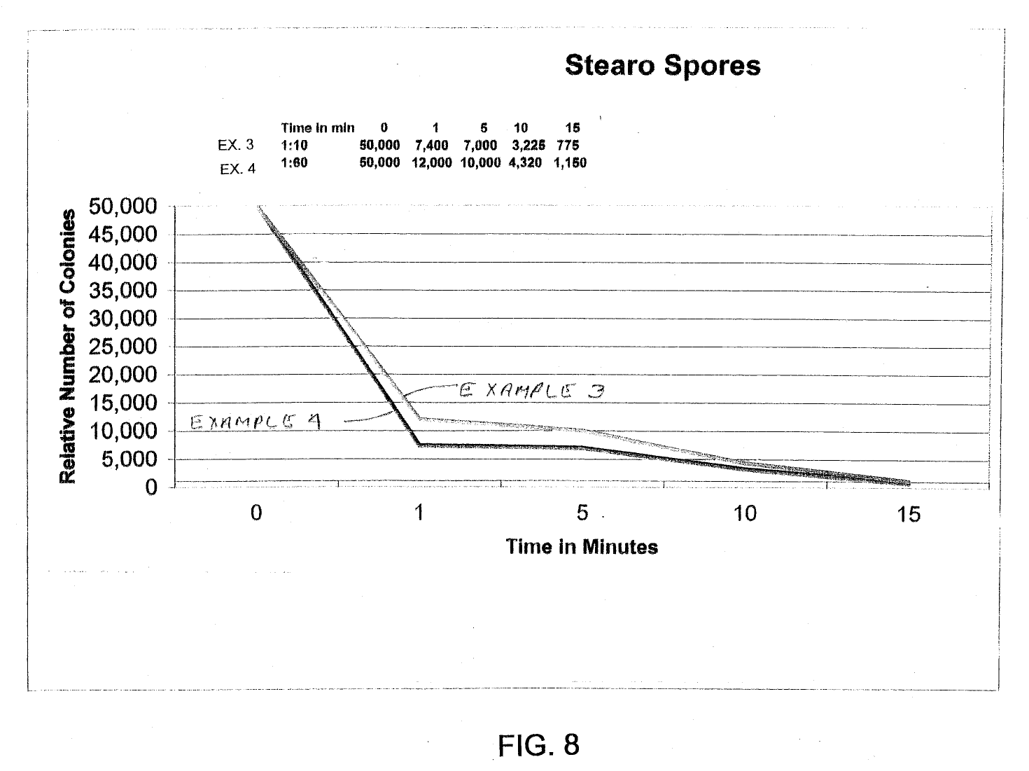

Referring to FIG. 8, the effectiveness or kill of Examples 3 and 4 against Bacillus stearothermophilus is reported. As shown, substantial kill is achieved in about 1 minute and substantially complete kill occurs in 15 minutes.

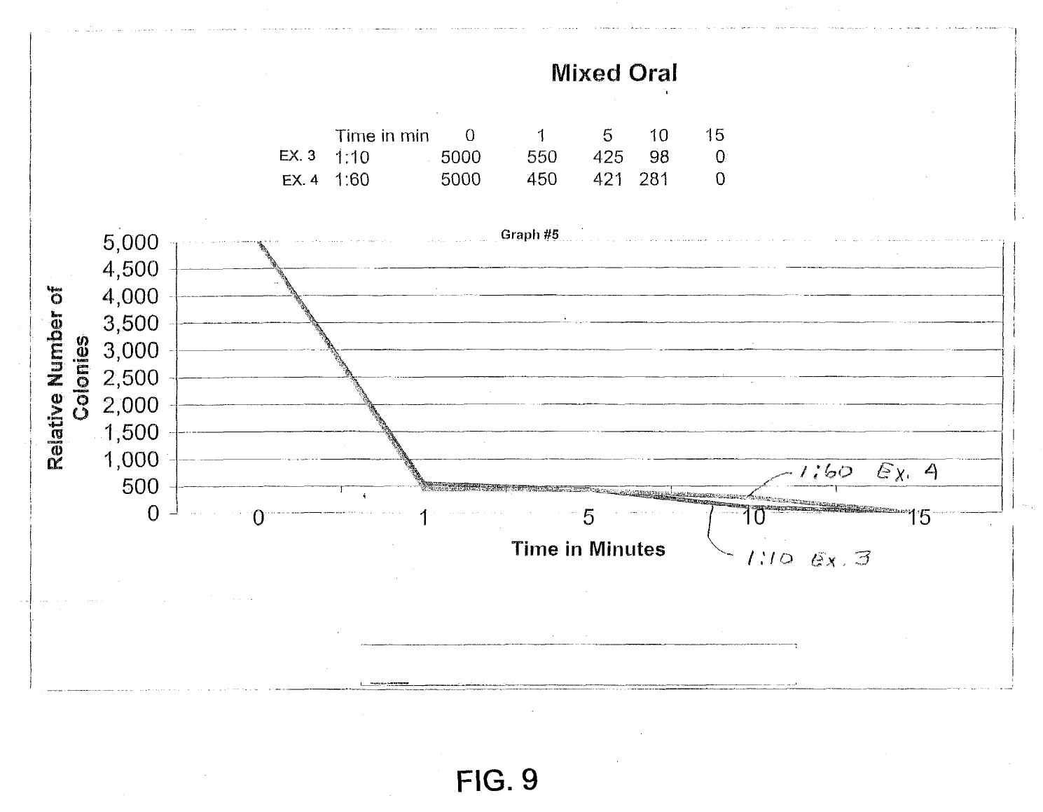

The effectiveness or kill rates of Examples 3 and 4 with respect to biofilms in the form of oral flora is reported in FIG. 9. The oral cavity is known to contain in excess of 500 species. This complex milieu containing clusters of plaque biofilm microbes takes about a minute to achieve a 99% kill and up to 10 minutes for complete eradication. However, there is again a general equivalence of effectiveness with substantial kill occurring in one minute and complete kill occurring in 15 minutes.

The compositions of the present invention have also been evaluated against Methicillin-Resistant Staphylococcus aureus (MRSA). MRSA is a type of Staphylococcus aureus that is resistant to antibiotics called beta-lactams. Beta-lactam antibiotics include Methicillin and other more common antibiotics such as oxacillin, penicillin and amoxicillin. Staph infections, including MRSA, are most frequently found among persons in hospitals and health care facilities, such persons having weakened immune systems. These Healthcare-associated staph infections include surgical wound infections, urinary tract infections, bloodstream infections and pneumonia. However, staph and MRSA infections can also cause illness in persons outside of hospitals and health care facilities. (See CDC MRSA Public Info.)

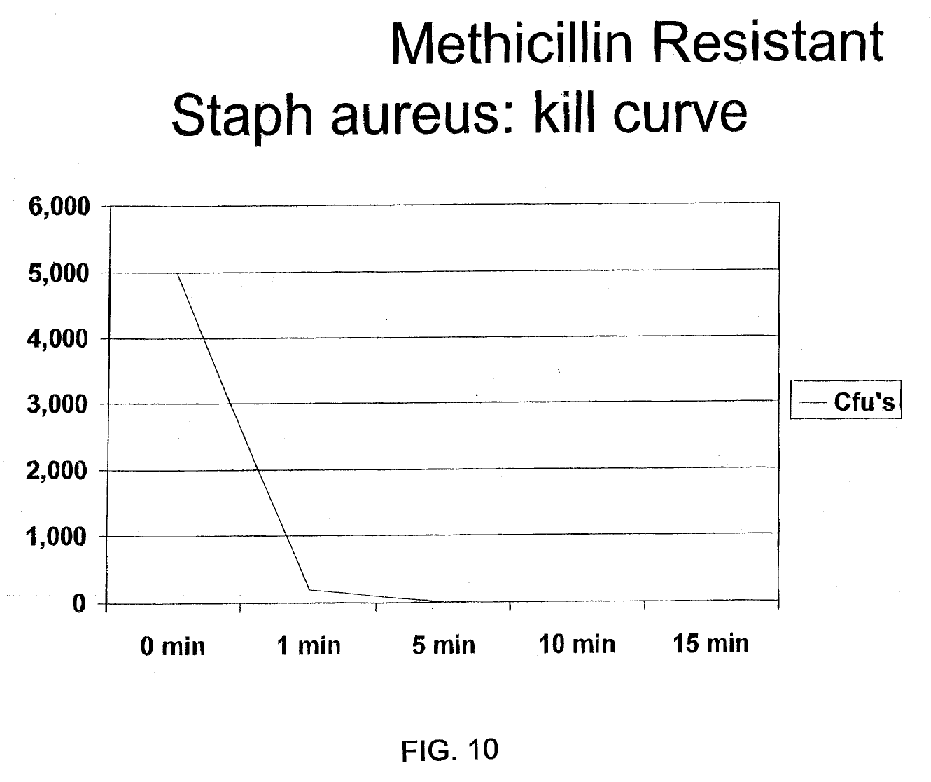

Using the same procedures as described above, the composition of Example 2, diluted with distilled water to a of 1:20 dilution, is combined with a one billion cfu sample of MRSA at room temperature. The MRSA kill rate over time is reported in the graph of FIG. 10. As shown, substantial kill occurs in about one minute and substantially complete kill occurs in less then about 8 minutes with a concentration of about 1.25% by weight.

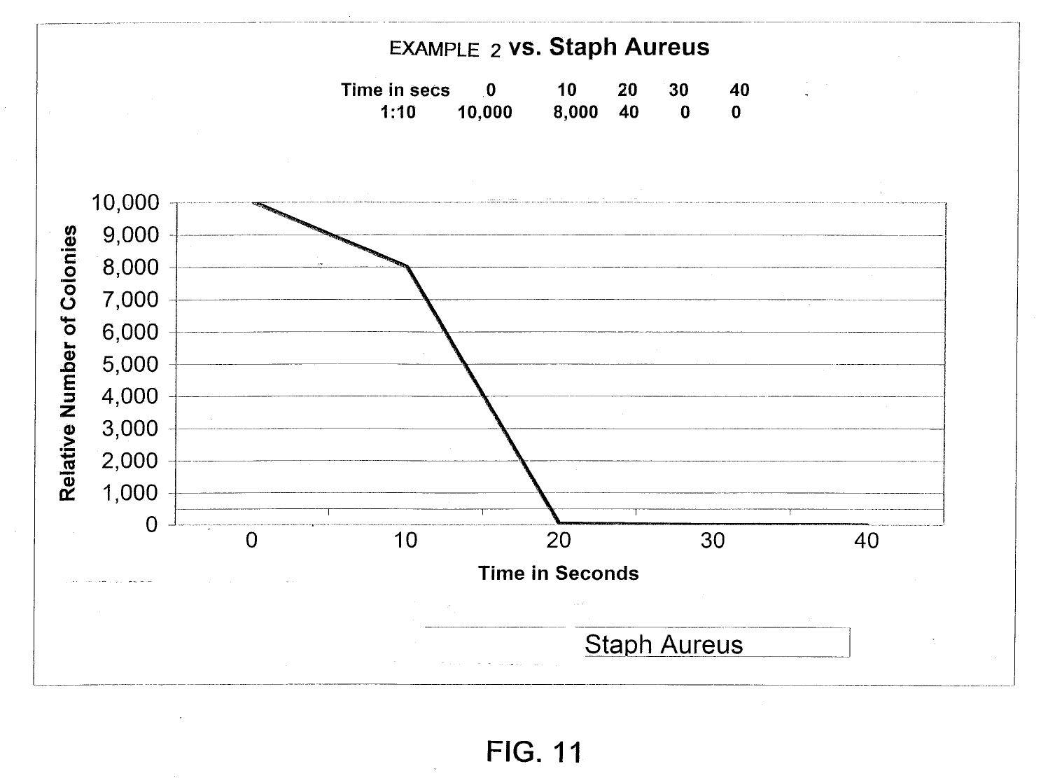

The compositions of the present invention have also been evaluated against Staphylococcus aureus to demonstrate the rapid kill achieved. Using the same procedures as described above, the composition of Example 2, diluted with distilled water to a 1:10 dilution, is combined with a one billion cfu sample of Staph aureus at room temperature. The Staff aureus kill rate over time is reported in the graph of FIG. 11. (In FIG. 11, the scale is arbitrarily set for a 10,000 cfu start to demonstrate reduction even though a one billion cfu sample is present at time zero.) As shown, substantial kill occurs in about ten seconds and substantially complete kill occurs in less then about 30 seconds with a concentration of about 1.25% by weight.

The compositions of the present invention have also been evaluated against Acinetobacter baumannii which is a species of gram-negative bacteria commonly found in water and soil. During 1963-2003, A. baumannii became an increasingly important cause of nosocomial infections, particularly in ICU's. Treatment of infections attributed to A. baumannii can be difficult because the organism has intrinsic resistance to certain antimicrobial agents and has acquired resistance to many others. An increasing number of A. baumannii bloodstream infections in patients in military medical facilities involving service members injured in the Iraq/Kuwait region has been observed. The number of these infections and their resistance to multiple antimicrobial agents underscore the importance of infection control during treatment in combat and health-care settings, and the need to develop new antimicrobial drugs to treat these infections. CDC, MMWR, Weekly, Nov. 19, 2004/ 53(45); 1063-1066.

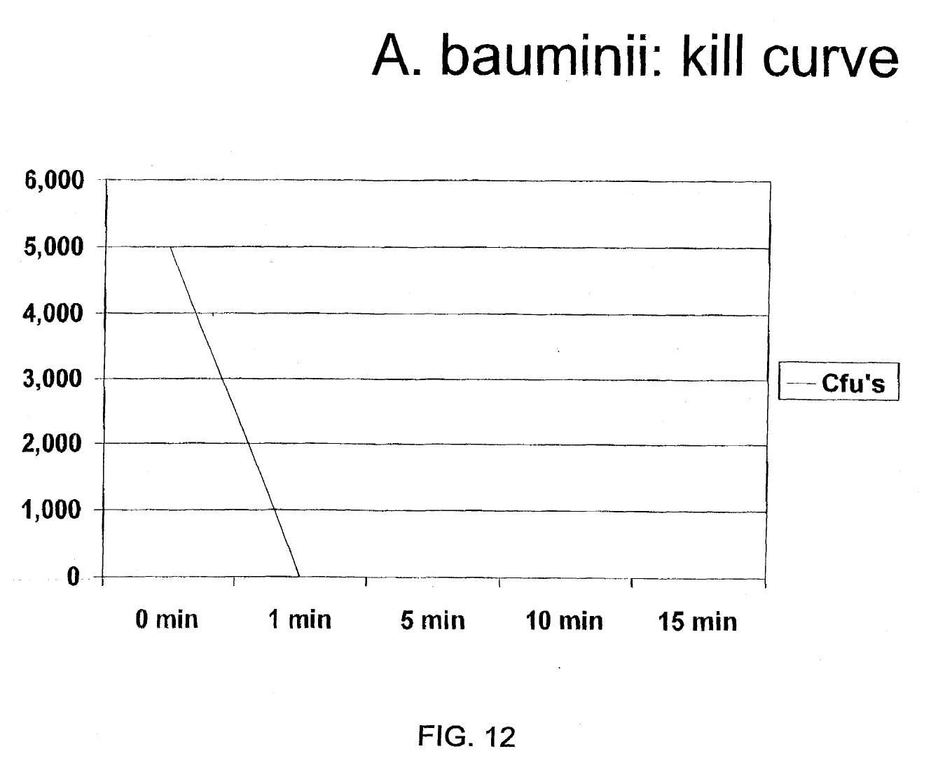

Using the same procedures as described above, the composition of Example 2, diluted with distilled water to a 1:40 dilution, is combined with a one billion cfu sample of A. baumannii at room temperature. The A. baumannii kill rate over time is reported in the graph of FIG. 12. As shown, substantial kill occurs in about one minute and substantially complete kill occurs in less then about 3 minutes with a compositions concentration of about 0.63% by weight.

The compositions of the present invention have also been evaluated against Vancomycin-resistant Enterococci (VRE). There are two types of Vancomycin resistance, namely, inherent and acquired. It is believed that Enterococci can become resistant to vancomycin by acquisition of genetic information from another organism. Rice, Emerging Infective Diseases, Vol. 7, No. 2, March-April 2001.

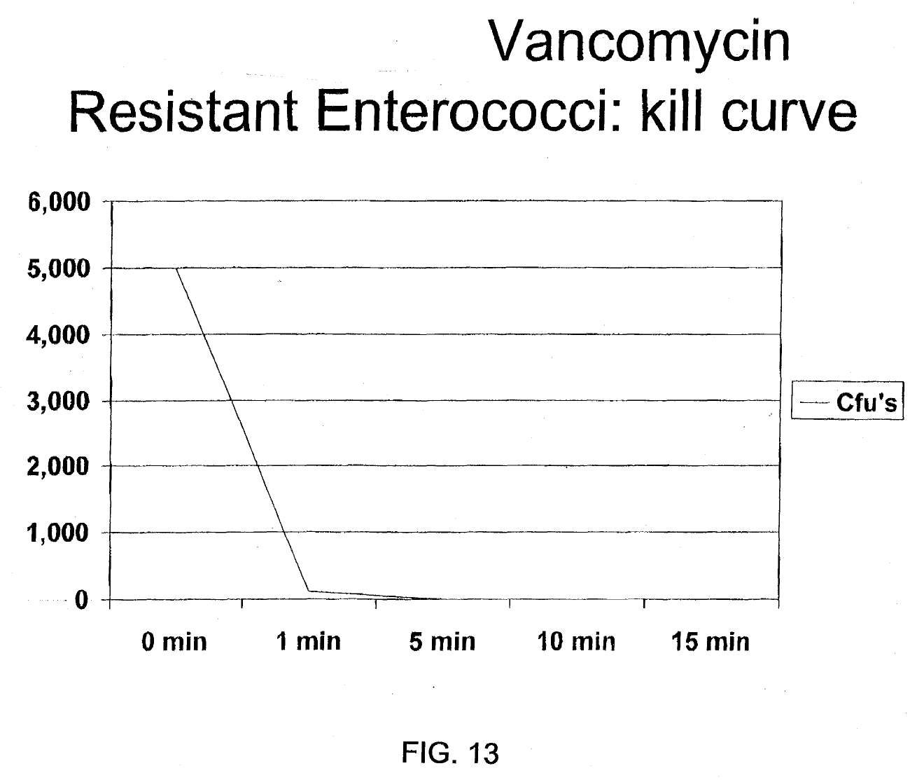

Using the same procedures as described above, the composition of Example 2, diluted with distilled water to a 1:20 dilution, is combined with a one billion cfu sample of VRE at room temperature. The VRE kill rate over time is reported in the graph of FIG. 13. As shown, substantial kill occurs in about one minute and substantially complete kill occurs in less then about 3 minutes with a concentration of about 1.25% by weight.

The compositions of the present invention are useful as disinfectants, such as Betadyne antiseptic and microbiocidal, and may be used in similar manners. In addition to Betadyne, commercially available disinfectants used in health care facilities include Vitaphene, Povidone Iodide, Aerocide, Cidex and Sporocidium.

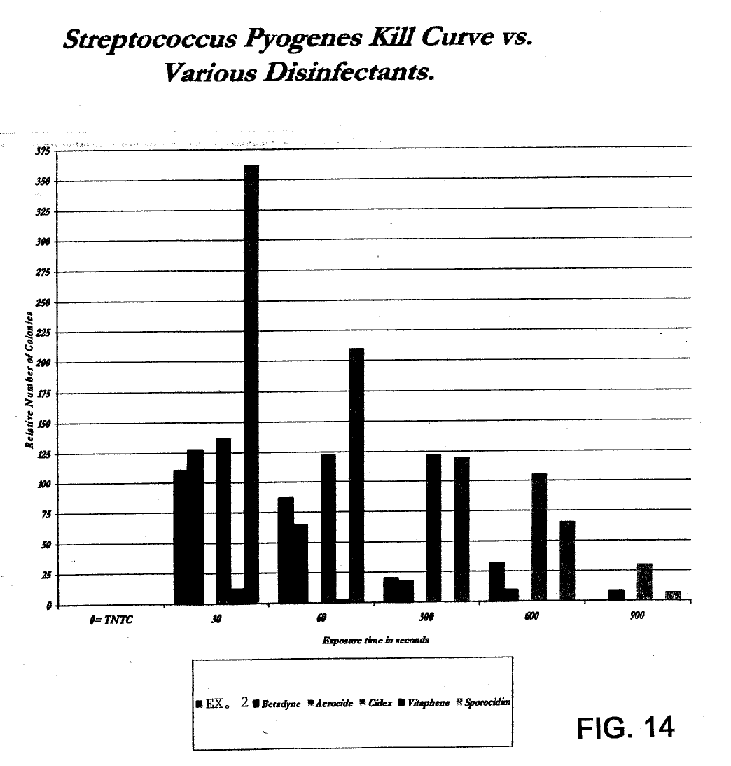

A comparison of the effectiveness of each of the foregoing against Streptococcus pyogenes is reported in FIG. 14. These disinfectants are comparatively evaluated herein at their commercially supplied concentrations. In each case, a 2 ml dose of 10⁸ Streptococcus/ml was tested against 200 microliters of the disinfectant. As shown, time points were measured in seconds up to 900 seconds. In all instances, Example 2 was as good, if not better, than the other disinfectants.

The disinfectants were also tested against mixed oral bacteria. The same dosage as described above was prepared of the oral bacteria and it was tested against a 200 micrometer sample of the disinfectant. In this instance, only Betadyne and Vitaphene approach the effectiveness of the composition of Example 2. The results are reported in the following Table 2.

TABLE 2

| 0 Sec. | 30 Sec. | 60 Sec. | 5 Min. | 10 Min. | 15 Min. | |
|---|---|---|---|---|---|---|
| TNTC | 0 | 0 | 0 | 0 | 0 | Example 2 |
| TNTC | 15 | 0 | 0 | 0 | 0 | Betadyne¹ |
| TNTC | 8,000 | 750 | 605 | 550 | 470 | Aerocide² |
| TNTC | TNTC | 9,000 | 8,000 | 7,200 | 1,275 | Cidex³ |
| TNTC | 25 | 3 | 0 | 0 | 0 | Vitaphene⁴ |
| TNTC | TNTC | TNTC | TNTC | TNTC | 8,000 | Sporocidium⁵ |

¹Betadyne a 10% iodide solution by Purdue Products L.P.
²Aerocide an o-phenylphenol 0.10%, 4-chloro-2 cyclopentylphenol 0.08%, lauric diethanolamide 0.20% and triethanolamine dodecyl benzenesulfonate 0.33% by G F Health Products, Inc.
³Cidex a 2.4% gluteraldehyde by Johnson and Johnson Div. of Ethicon of Irving, CA.
⁴Vitaphene a 9.0% alpha phenylphenol and 1.0% o-phenylphenol by Block Drug Corporation, Jersey City, NJ
⁵Sporocidium a 1.56% phenol, 0.06% sodium phenate by Sporicidin International, Rockville, MD.
TNTC means “too numerous to count”.

The compositions of the present invention are also useful for infection control in the antiseptic care of incisional and burn wounds. Wound contamination and the subsequent decontamination of wounds is of interest in a combat care setting. A number of methods are currently in use in wound and instrument decontamination including sterilization, disinfection, and antisepsis.

Contamination is defined as the introduction of microorganisms into tissues or other materials, whereas decontamination is defined as the reverse. That is, disinfection or sterilization of infected wounds to an acceptable level (noninfectious level).

The efficacy of the compositions of the invention against human pathogenic bacteria was evaluated. For this purpose, evaluation of the bacterial “kill” on uncompromised normal skin was evaluated. In these experiments, bacterial strains of Staphylococcus aureus, Pseudomonas aeruginosa and normal oral flora were introduced to the shaved backs of rabbits in concentrations of 1×10⁹ cfu/25 ul. Following application of the bacterial treatments, a saline control, Betadyne and the composition of Example 2 were applied at a rate of 100 ul per square inch. Betadyne and Example 2 were found to prevent bacterial growth, that is, they showed similar results in the limiting of the growth of the applied bacteria and ultimately killing the bacteria.

The antiseptic effect of treatment with saline, Betadyne and Example 2 in a partial thickness incision model was also evaluated. A 2.5 cm incision extending through the dermal layer was made in the shaved backs of New Zealand White rabbits. As in the clear skin studies, the various microbes at similar concentrations were placed in the incisions and the site treated with 100 ul of saline, Betadyne or the composition of Example 2. The incisions were covered with occlusive Hilltop chamber dressings. Once again, the Betadyne and the composition of Example 2 showed like inhibition and kill of the bacteria.

In the evaluation of burn wounds, the antiseptic properties of Betadyne and the composition of Example 2 were compared against saline control. In the burn model, the wound is created and the bacteria applied to the healing wound as would be the case in the field. In this instance, the shaved backs of guinea pigs were burned and covered with an occlusive Hilltop chamber dressing for 24 hours. Thereafter, the burn wound is debrided and intentionally infected with bacteria as described above. Once again, the antiseptic properties of Betadyne and Example 2 were comparable.

The composition of Example 2 is as effective as Betadyne in the decontamination of intentionally contaminated clear skin, incisions and partial thickness burns.

Additional microbes of particular interest were evaluated in further rabbit studies. The additional test microbes included:
Methicillin Resistant Staph aureus (MRSA)
Strep Pyogenes
Vancomycin Resistant Enterococci faecalis (VRE)
E. coli
Pseudomonas aeruginosa
   After the animals were anaesthetized and shaved as previously described above, a deep wound was made on each side of the backs of the animals with a scalpel. One of the wounds was for Betadyne and one wound was for Example 2. Microbial cultures grown on blood agar were inoculated heavily on cotton swabs directly from large colonies and rubbed into the wound sites. Inoculation of the wounds was estimated by examination of comparable cotton swabs which had their contents dislodged by sonication or high-speed circular spin procedures to suspend and isolate bacteria from the cotton swab tip. The microbes isolated in suspension were diluted by 10 fold dilution procedures and counted in pour plates of Trypticase soy agar, yeast extract and Todd-Hewitt broth (10:1:5). The cfu account revealed a range of 100-120 million cfu's/swab. Therefore, a direct inoculation of about 110 million bacteria were swabbed directly into rabbit wounds. Massive inocula were therefore achieved. Distinctive colonies were stained for morphology and gram staining characteristics.

The following day, two milliliter doses of Betadyne and Example 2 were respectively applied at room temperature by dropper at various points. The composition of Example 2 was applied at a 1:10 dilution (2.5% by weight concentration of active ingredients) in two milliliter doses by drop wise application to the wound. Swabs were taken after one minute, five minutes and one hour to determine cfu's remaining on the wound. This was followed by a three-day waiting period with no additional disinfectant applied.

Swabs taken from the animals were placed in 3 ml saline and vortexed for 30 seconds to remove bacteria. Samples were spread by plastic spreaders on blood agar and incubated for 48 hours for cfu analysis. The results are reported below Table 3.

TABLE 3

| | | Strep | | | |
|---|---|---|---|---|---|
| | MRSA | Pyogenes | VRE | E coli | Pseudomonas |
| Baseline | 378 | 217 | 212 | 406 | 397 |
| 1 min. | 335 | 220 | 243 | 356 | 350 |
| Betadyne | | | | | |
| 1 min. | 135 | 117 | 36 | 200 | 300 |
| Example 2 | | | | | |
| 5 min. | 286 | 65 | 60 | 165 | 112 |
| Betadyne | | | | | |
| 5 min. | 36 | 17 | 10 | 40 | 56 |
| Example 2 | | | | | |
| 24 hrs. | 585 | 305 | 393 | 375 | 428 |
| no | | | | | |
| further | | | | | |
| treatment | | | | | |

As indicated by the data, treatment with Example 2 after one minute shows some microbe reduction. However, there is little effect, if any, for Betadyne. After five minutes, good reduction of all five microbes is found with Example 2. In comparison, fair to good reduction is also found with Betadyne after this passage of time.

After 24 hours, the microbes reestablish themselves indicating that the multiple doses of disinfectants should be applied over the course of several days for wound healing, surgical intervention or other treatment. Multiple applications or continuous contact with the inventive compositions, which are both possible due to its low toxicity level, would keep the wound in an excellent stage for healing and/or subsequent surgery.

The efficacy of the inventive compositions in respect to naturally encountered microbes in various soils was also investigated. To that end, soil samples were taken from the following locations.
Desert—the Mojave Desert, Calif., 60 miles north of the City of Mojave and 20 miles south of Adelanto. Altitude 2,300 feet above sea level.
Mountain—The Sierra Nevada Mountain Range, McGee Canyon, Calif., 13 miles south of the town of Mammoth Lakes at 7,600 feet above sea level.
Beach—A beach on the island of Kauai, Hi., 12 miles north of the airport at sea level elevation.

The collected soil samples were weighed out into 2 gram aliquots. The aliquots were suspended in 15 ml of sterile water, shaken into suspension and a 1 ml water suspension sample removed. The 1 ml aliquot was pipetted and a dilution series made twofold. Enriched agar media was poured into petri dishes and counted after three days incubation at ambient temperature.

Samples of 1.0 ml aliquots were treated with a 0.1 ml aliquot of Example 2 diluted 1:10 to test microbial kill. In various time increments, 0.5 ml aliquots were pipetted into petri dishes and 12 ml of enriched nutrient agar was added. The sample was allowed to solidify and measured after three days for colony forming units cfu's. The results are reported in the following Table 4.

TABLE 4

| Time (min.) | Desert | Mountain | Beach |
|---|---|---|---|
| 0 | 850 | 1,100 | 6778 |
| 1 | 76 | 52 | 26 |
| 2 | 0 | 0 | 0 |
| 5 | 0 | 0 | 0 |
| 10 | 0 | 0 | 0 |
| 15 | 0 | 0 | 0 |

The results reported in Table 4 show that the composition of Example 2 killed all microbes isolated from soil samples obtained from desert, mountain and beach soils or sands. The complete kills were obtained within two minutes, where as, about 90% kill or better, was obtained in the first minute of contact with Example 2.

The effectiveness of the compositions of the present invention in connection with the regulation of bacterial biofilms was evaluated in connection with Staph aureus, Pseudomonas aeruginosa, MRSA, mixed oral bacteria, Enterococci faecalis and E. coli. In each instance, a mature and healthy biofilm was cultivated on a gel surface to provide a matrix size of about a square inch or more. The starting biofilm was three days old and grew as an amorphous smooth surface gel-like mass owing to the mucous secretion of the adherent mass of bacteria.

Each biofilm sample was contacted with the composition of Example 2 at room temperature and at a rate of 10 ml per sample for three and 15 minutes treatments. After the treatment times, the biofilms were washed with phosphate buffered saline and fixed with gluteraldehyde. They were then prepared for scanning electron microscope (SEM) without otherwise affecting the nature of the test.

The following observations characterize the effectiveness of the composition to control and destroy the biofilm mass with kill of the bacteria species.

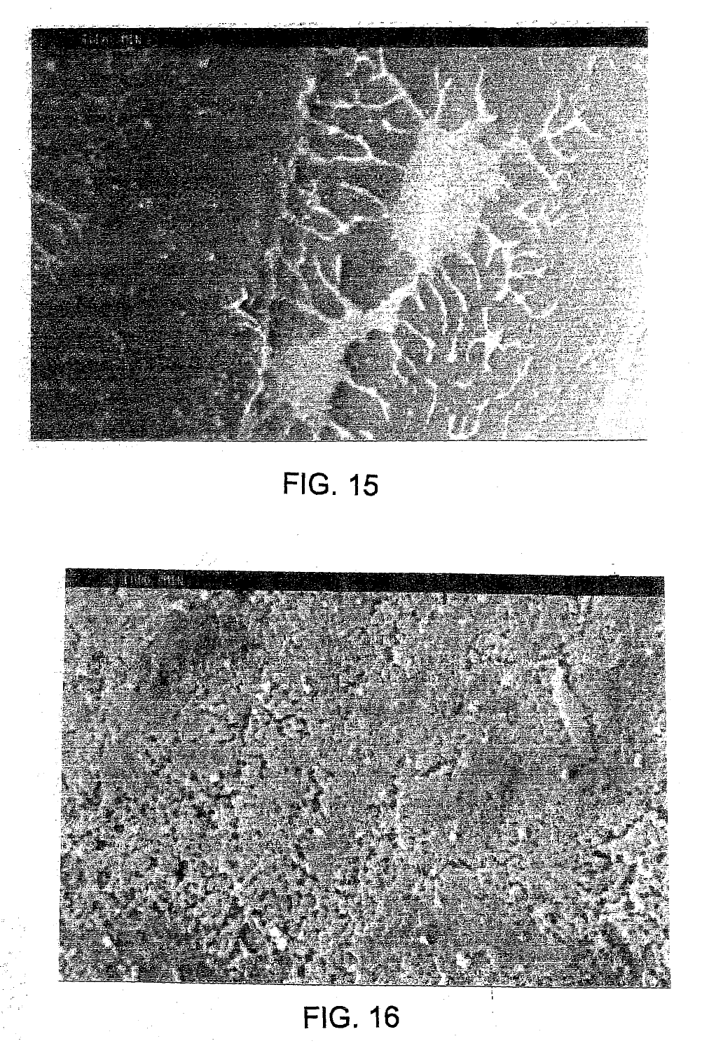

FIG. 15 shows the Staph aureus biofilm after three minutes treatment with Example 2 as seen at 100× magnification. In this instance, the composition was effective to dissolve the biofilm for the most part, and the bacteria were reduced to a planktonic state after 15 minutes, but not killed.

FIG. 16 shows the Pseudomonas biofilm after three minutes treatment with Example 2 as seen at 2000× magnification.

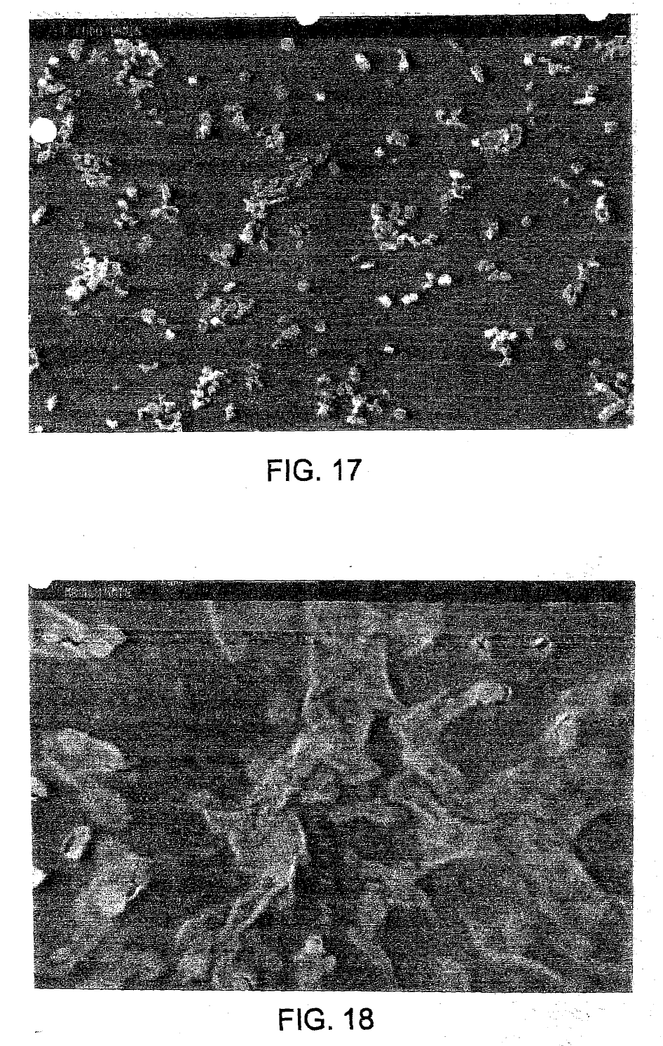

FIG. 17 shows the Pseudomonas biofilm after 15 minutes treatment with Example 2 as seen at 2000× magnification. Considerable damage and substantially complete kill has occurred to the biofilm.

FIG. 18 shows the MRSA biofilm after 15 minutes treatment with Example 2 as seen at 5000× magnification. A complete destruction of the bacteria in the biofilm is shown. The matter in the photomicrograph is the leftover slime that once covered the biofilm colony.

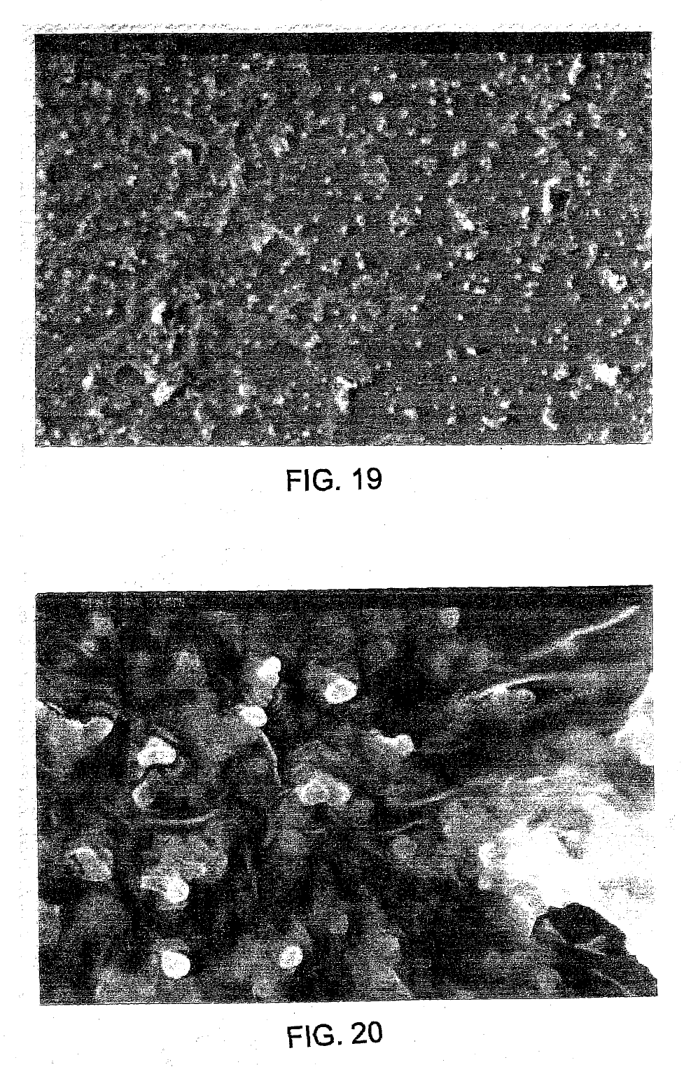

FIG. 19 shows the mixed oral biofilm after 3 minutes treatment with Example 2 as seen at 1000× magnification. As shown, the biofilm colony has been broken with parts reduced to a planktonic form. About one-half the biofilm was reduced to the planktonic state with very little bacterial kill.

FIG. 20 shows the mixed oral biofilm after 15 minutes treatment with Example 2 as seen at 5000× magnification. A large part of the colony has been unaffected. About one-half the biofilm was reduced to the planktonic state with very little bacterial kill.

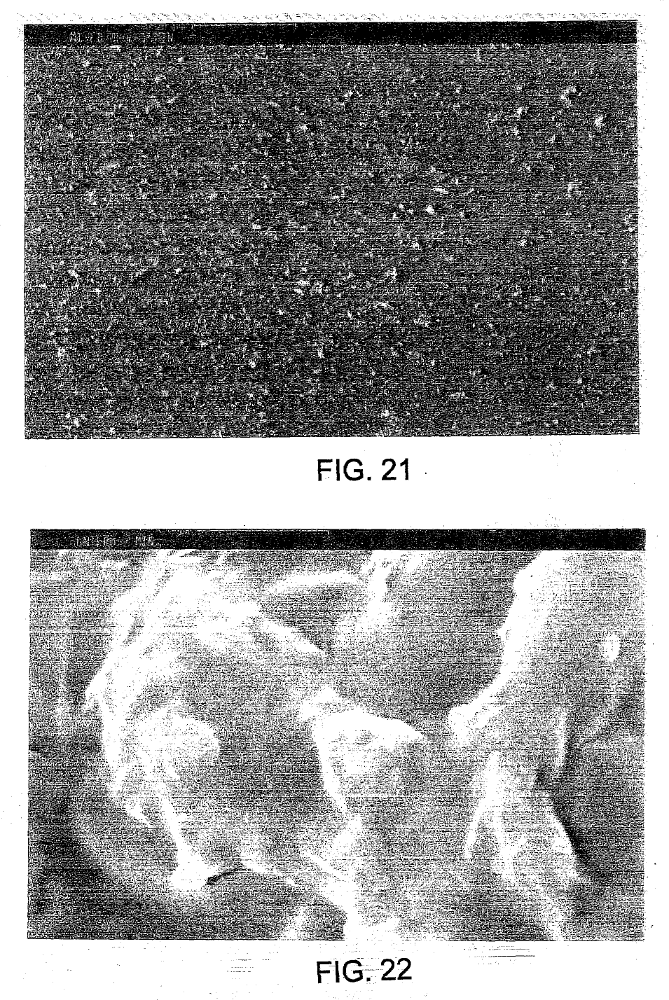

FIG. 21 shows the mixed oral biofilm of FIG. 21, but at 100× magnification to give a broader view.

FIG. 22 shows the Enterococci biofilm after 3 minutes treatment with Example 2 as seen at 5000× magnification. About one-half of the biofilm was destroyed. The remains of the biofilm slime are shown devoid of any bacteria.

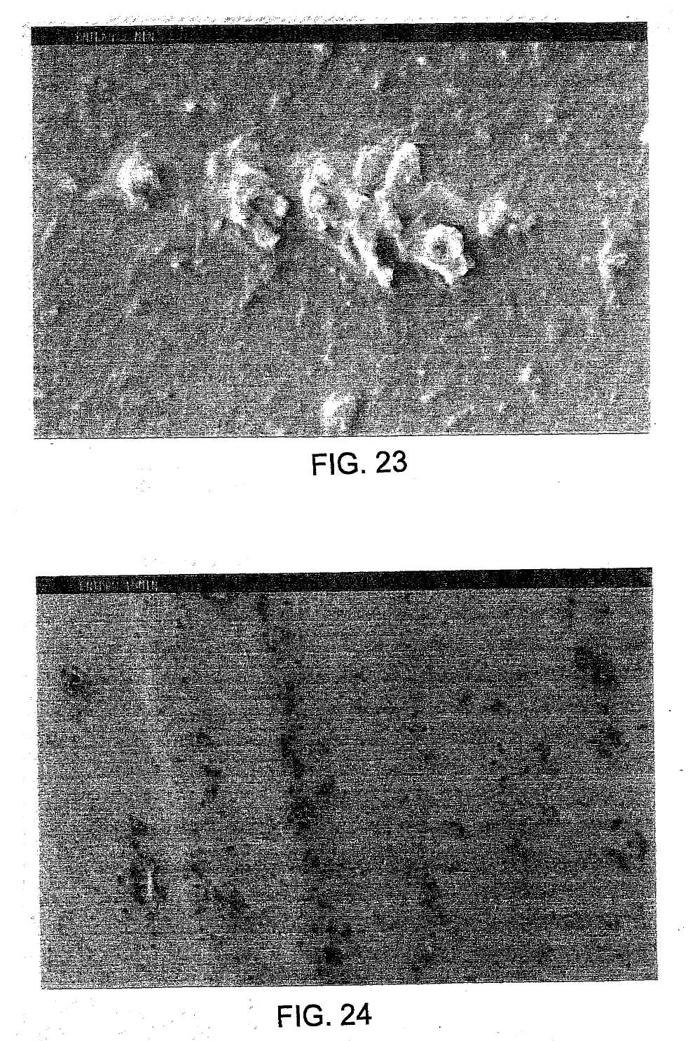

FIG. 23 is similar to FIG. 23, but shows another part of the remains of the biofilm as seen at 1,100× magnification.

FIG. 24 shows the complete destruction of the Enterococci biofilm after 15 minutes treatment with Example 2 as seen at 100× magnification.

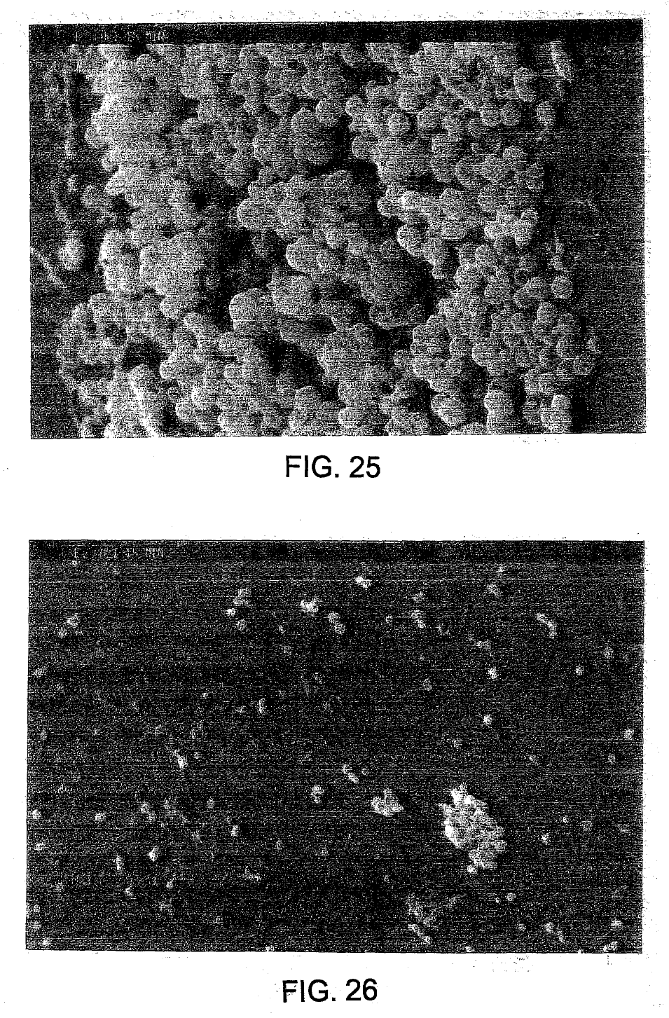

FIG. 25 shows the E. coli biofilm after 15 minutes treatment with Example 2 as seen at 5000× magnification. A noticeable breakup of the biofilm colony is noticed in three minutes and after 15 minutes the E. coli colony has been taken out of its biofilm state.

FIG. 26 shows the E. coli biofilm after 15 minutes treatment with Example 2 as seen at 2,000× magnification.

The compositions of the present invention are also useful for personal hygiene, as for example, a liquid soap composition. In liquid form, the composition may be dispensed using a conventional pump arrangement and a plastic container. To that end, the composition of Example 2 was evaluated as a soap and a shampoo to demonstrate successful reduction in microbe count in key body areas, such as the head, face, legs, arms and feet.

Test individuals included four males ranging from 17 to 66 years of age. One female was tested for hand cleaning. The inventive compositions were compared with the following commercial products.

1) DOVE brand white bar soap by Unilever of Turbil, Conn., USA, and

2) Liquid antibacterial soap sold under the DIAL trademark by the Dial Corporation of Arizona, USA.

3) KIRKLAND brand shampoo marketed by Costco Corporation of Seattle, Wash., USA. This shampoo contains sodium lauryl sulfate, cocamidopropyl betain, aloe vera, jojoba oil, methylparaban EDTA, methylchloroisothiaolilnone and algal extract.

There is considerable variability in individual washing procedures. This includes both body wash and shampoo applications. In spite of such variation, the compositions were found to reduce microbial levels from every test site. The sites of highest microbe loads were hairy areas such as the chest, under arms and groin.

Baselines were established by swabbing at the end of the workday or in the morning. Swabs were inoculated directly on blood agar plates, or in the case of high counts, swabs were broken off in test tubes with 5 ml sterile saline, and mixed in a vortex mixture for 2 minutes to release bacteria from the swabs. Aliquots were then measured by dilutions and 0.5 ml was added to a blood agar plate. The mixture was spread by a plastic plate spreader and incubated for 48 hours prior to plate counts and cfu determinations.

TABLE 5
Head and Upper Body (cfu count)

| | Start | After Soap/ | After |
|---|---|---|---|
| | Baseline | Shampoo | Example 2 |
| Cheek | 489 | 312 | 67 |
| Nose | 3,027 | 2,905 | 190 |
| Chin | 110 | 80 | 56 |
| Hair | 212 | — | 57 |
| Scalp | 2,100 | — | 222 |
| Forearm | 57 | 32 | 11 |
| Bicep | 25 | 20 | 10 |
| Underarm | TNTC | 4,050 | 3,450 |

Using the foregoing procedures, additional evaluations were made as reported below in Table 6.

TABLE 6
Hair and Body Parts (cfu count)

| | Start | After Soap/ | After |
|---|---|---|---|
| | Baseline | Shampoo | Example 2 |
| Chest | 1,125 | 385 | 101 |
| Back | 127 | 96 | 55 |
| Buttocks | 17 | 12 | 6 |
| Thigh | 121 | 26 | 11 |
| Calf | 47 | 29 | 12 |
| Foot | 98 | 80 | 48 |
| Between Toes | TNTC | TNTC | 3,080 |
| Back of Hand | 47 | — | 1 |
| Palm | 212 | — | 2 |
| Hand Knuckle | 10 | — | 1 |
| Hand Nail | 1,600 | — | 960 |
| Thumb | 6 | — | 1 |
| Forefinger | 12 | — | 3 |

The compositions of the present invention are useful in connection with instrument sterilization in the field. Instruments tested included scissors, forceps, tweezers, dental burs, probes, explorers and clamps. Serrated edges, hinged devices and knurled ends were particularly examined to confirm whether sequestered areas could be disinfected.

The instruments were placed in trays containing 10⁸ bacteria per milliliter and allowed to remain in contact for 45 minutes. The instruments were then removed, air-dried, and placed in sterile tubes with various dilutions of Example 2 including 1:5, 1:10, 1:20 and 1:40. After incubating with Example 2 for various times, the instruments were removed, dipped in saline, and placed aseptically in sterile tubes of appropriate sizes containing sterile media and incubated at 35° C. for up to 8 days.

Tubes and positive controls could be visually detected by turbidity. Media containing purple base could be detected by observing a purple to yellow color shift via pH change by acid production indicating microbial growth. Growth was surveyed at room temperature and at 35° C. incubator temperature under aerobic conditions.

Positive control tubes showed turbidity at 24 hours and extensive turbidity at 48 hours. Under proper conditions, no growth was observed at eight days. In some conditions of lower-level kill at eight days, very few microbes per milliliter were detected, the worst case scenario being less than 10 microbes were found. Under the sterilization conditions, no turbidity or pH change is detected, nor any cfu's noted when 1 ml of test media was inoculated and spread on the surface of blood agar plates.

In the following Tables 7, 8 and 9, the reduction Strep pyogenes at day 8 after exposure to Example 2 for various times is reported.

TABLE 7
Reduction of Strep Pyogenes
Day 8 of Test After 5 minute Exposure to Example 2

| | Turbidity | pH Shift | cfu's |
|---|---|---|---|
| −Control | None | None | 0 |
| +Control | Heavy | Yes | TNTC |
| 1:5 Dilution | 0/3 | Slight | 63 |
| 1:10 Dilution | 2/3 | Moderate | 3,050 |
| 1:20 Dilution | 3/3 | Heavy | TNTC |

TABLE 8
Reduction of Strep Pyogenes
Day 8 of Test After 10 minute Exposure to Example 2

| | Turbidity | pH Shift | cfu's |
|---|---|---|---|
| −Control | None | None | 0 |
| +Control | Yes | Yes | TNTC |
| 1:5 Dilution | None | None | 0 |
| 1:10 Dilution | Slight | Yes | 693 |
| 1:20 Dilution | Yes | Yes | 7815 |

TABLE 9
Reduction of Strep Pyogenes
Day 8 of Test After 15 minute Exposure to Example 2

| | Turbidity | pH Shift | cfu's |
|---|---|---|---|
| −Control | None | None | 0 |
| +Control | Yes | Yes | TNTC |
| 1:5 Dilution | 0 | 0 | 0 |
| 1:10 Dilution | ± | Slight | 16 |
| 1:20 Dilution | + | + | 1720 |

The foregoing data confirm that the composition of Example 2 is capable of disinfecting as long as sufficient time elapses for contact with the contaminated instrument. Presently, it appears that a minimum of about 15 minutes is required for complete disinfection to occur. For convenience, a device impregnated with Example 2 may be contacted with the instrument to maintain constant contact during the procedure. A moist liquid bandage of the composition provides optimum results. For example, the instrument may be wrapped with a foraminous or fibrous carrier material impregnated with the composition and having an impermeable outer sealing layer.

It should be appreciated that the compositions themselves may be formed into integral bandages in situ. The compositions may be applied as a thin liquid film or as a foam and allowed to dry to a continuous thin film. For example, diluted compositions of Example 2 at concentrations ranging from about 2% to about 5% active ingredients will form a foam upon dispensing with mild agitation as resulting from hand the liquid from a container. Satisfactory results have been obtained with bottles marketed by Ainspray International Incorporated.

A measured pump volume of about 0.3 ml will typically treat a two to three inch long skin wound with a foamed layer of the composition resulting from direct pump-bottle application. The foam is temporarily sustainable at room temperature and atmospheric pressure. In a few minutes, the foamed composition spreads out and collapses to form a substantially continuous film or thin strip about 1 by 3 inches long. The thickness of the thin strip is estimated to be a few thousands of an inch.

A single bandage formed in this manner will last for one to two days, but the bandage may be applied two or more times daily. In two days, a typical cut wound is scabbed over. Initial tests indicate that the bandage is effective to prevent infection of wounds such as burns, glass or metal cuts or on a skin biopsy for a mole removal. It appears that rapid healing is promoted.

The compositions of the present invention are useful as antiseptics or disinfectants for treating of medical facilities per se. For example, over thirty medical, dental and laboratory facilities including operatories, laboratory equipment and waiting rooms were cleaned using the compositions in the form of foams, sprays and liquid as applied with a wipe cloth. The composition of Example 2 has shown excellent antimicrobial/cleaning powers at least equaling, but usually exceeding, other standard disinfectants.

The areas of highest contamination in dentistry were sinks, floors, high power evacuation lines and counter tops. Aerosol studies indicate that the higher the microbial count in water lines, the higher the surface count. Aerosol fallout is the source of surface contamination. Patients with high oral microbial counts also add greatly to the aerosol bioload during operative procedures.

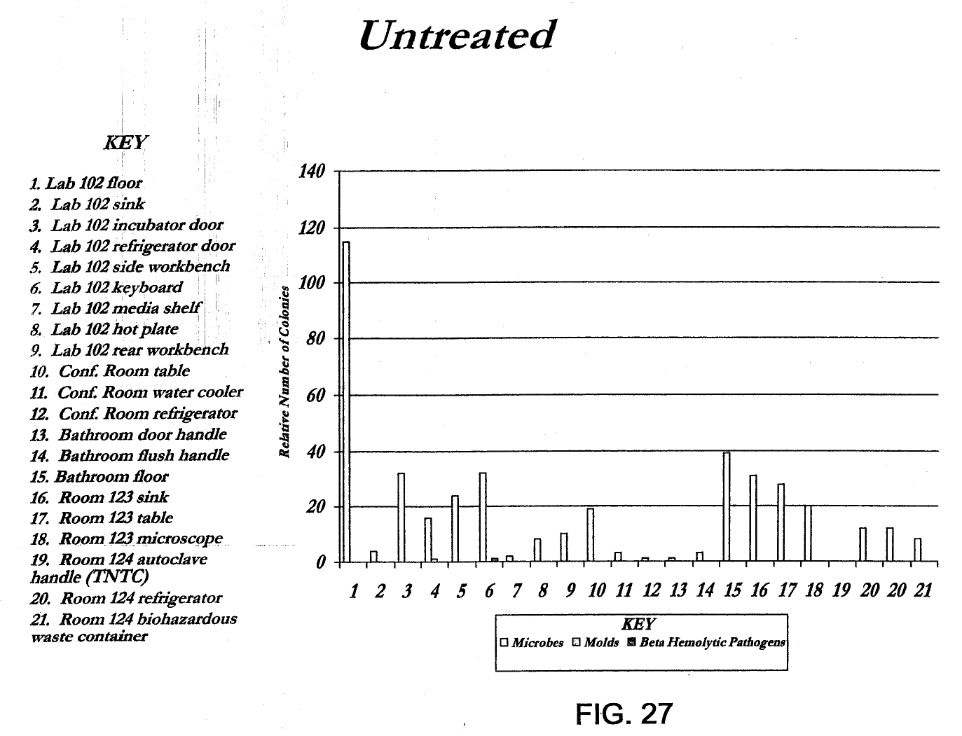

Referring to FIG. 27, a survey count of the microbes, molds and Beta hemolytic pathogens present in the indicated untreated areas of a tested dental facility is shown. The microbe count on the autoclave handle exceeded the report range, and next highest count of microbes occurred on the lab floor.

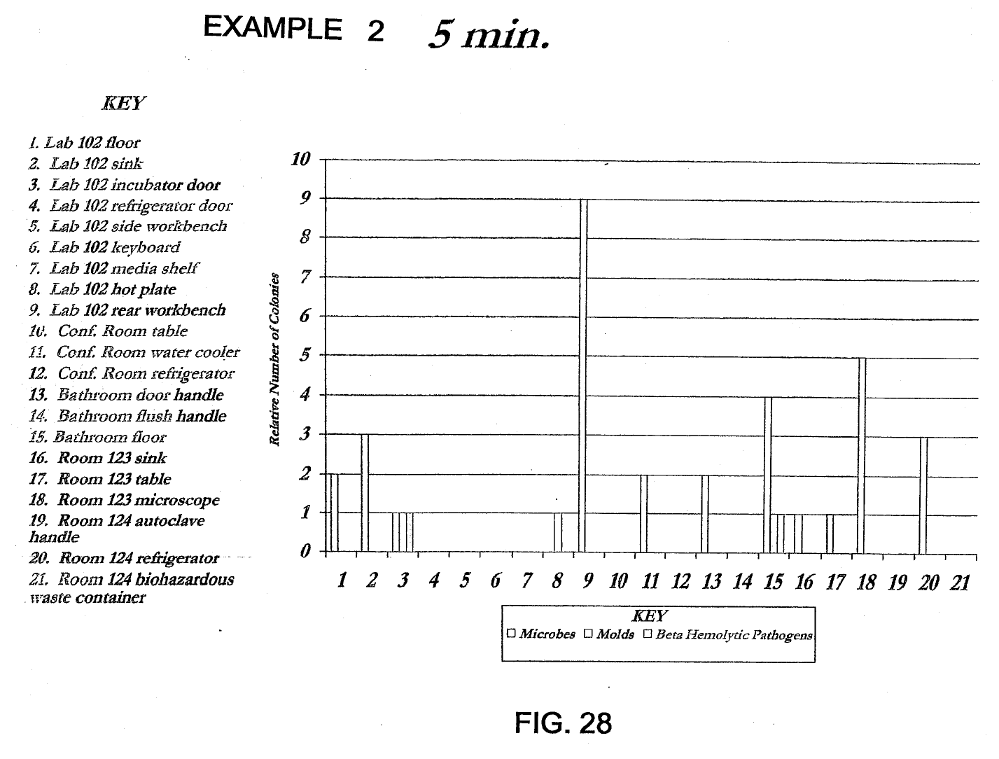

Referring to FIG. 28, a similar survey report of microbe count after five minutes following treatment with the composition of Example 2 is shown.

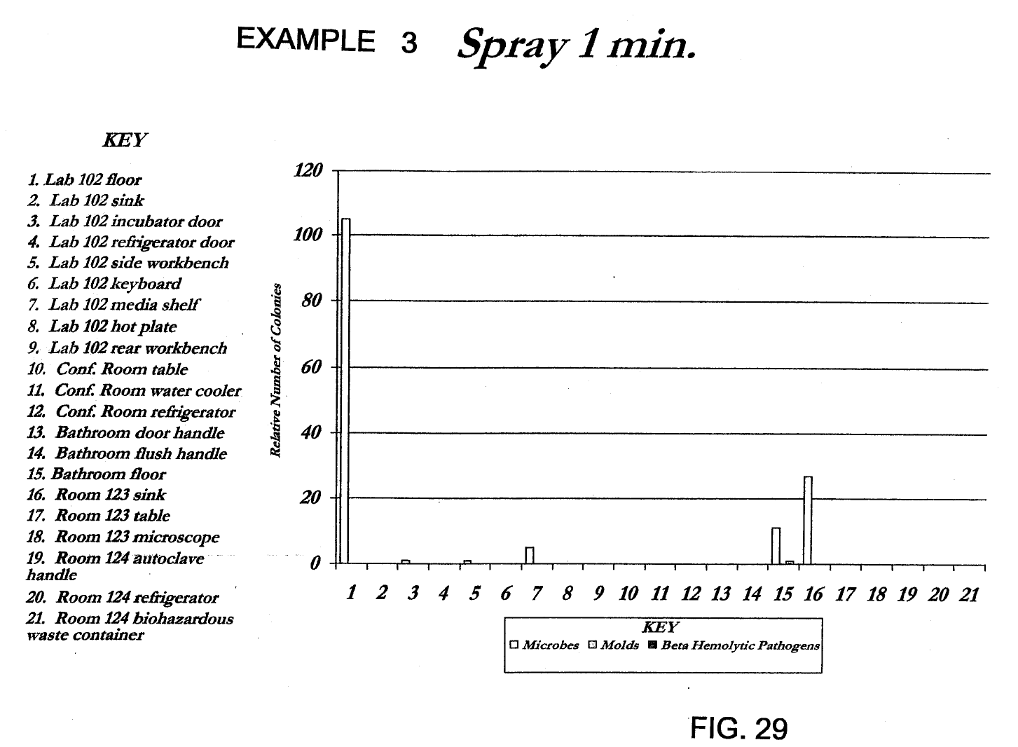

Referring to FIG. 29, a count of the dental facility is shown one minute after a spray application of a 1:10 dilution of the composition of Example 2. As indicated, a significant reduction in the microbe count occurs in all areas except for the lab floor.

The comparative use of bleach and the composition of Example 2 to clean the operatory lab and laboratory facilities with respect microbes, molds and pathogens is summarized in the following Table 7. As shown, Example 2 is as effective as bleach in reducing to substantially zero the microbe, mold and pathogen counts.

TABLE 7
MICROORGANISM COUNT

| | Untreated | Example 2 | Bleach |
|---|---|---|---|
| Operatory Lab | | | |
| Microbes | 5000 | 0 | 0 |
| Molds | 0 | 0 | 0 |
| Pathogens | 0 | 0 | 0 |
| Operatory | | | |
| Microbes | 5000 | 0 | 0 |
| Molds | 0 | 0 | 0 |
| Pathogens | 0 | 0 | 0 |

The compositions of the invention are also useful in connection with the operation and maintenance of dental hand pieces. The compositions may be added to the circulating water system for the dental hand piece to provide sterilization-disinfectant, antiseptic and lubricant properties during operation. Further, the severe conditions of the autoclave procedures heretofore used to sterilize dental hand pieces may be replaced by room temperature contact sterilization treatments with the inventive compositions. The foregoing use of the compositions significantly reduces expected maintenance repairs of the hand pieces.

The compositions may be added to a closed water circulation system for the dental hand piece to provide a fluid mixture having a concentration of active ingredients equal to about 0.1%. The fluid is circulated to the hand piece which impinges a stream of fluid onto the tooth surface being cut. Without detriment to the cooling effect of the fluid, the impinged fluid is dispersed and forms an antiseptic aerosol in the oral cavity with activities exemplified by the mixed oral flora tests reported in connection with FIG. 9. The sterilization effectiveness of the fluid is confirmed by cleaner evacuation traps for the water system believed to result from the inhibition of biofilm formation and reduced levels of microorganisms. The traps previously contained a gel-like biofilm, but the described use of the compositions results in a white powder in the traps that is believed to be the residue of the destroyed biofilms.

The compositions may be used at a concentration of active ingredients of about 1.0% to wash and soak the hand pieces in a room temperature sterilization process that replaces the previously used high temperature autoclave cycles. The hand piece is initially taken apart, sprayed with the composition to remove bulk debris and than allowed to set for 10 minutes. Thereafter, the sterilization is completed by soaking the hand piece in the composition for about 1 hour to about 1.5 hours at room temperature. But the hand piece may be left in the composition overnight for convenience and a total treatment of approximately 20 hours without adverse effect. This sterilization process is believed to extend the lives of the elastomeric gaskets and fiber optic tube components as compared with autoclave treated hand pieces.

The compositions have a lubricious quality that provides effective lubrication of the rotating components such as the turbine and its rotational mounting assembly in the hand piece. In a long term test including multiple low and high speed hand pieces, the incident of expected replacement of the turbine and chuck assembly was reduced by about 80%. That is, the seven hand pieces tested required replacement of six turbine and chuck assemblies during the test period. In comparison, it would have been expected to replace about 35 turbine and chuck assemblies in seven such hand pieces when used for a like duty cycle and time period with a water coolant and autoclaving in accordance with prior art procedures.

The compositions of the present invention are not toxic and do not result in cell damage at useful pH values in the range of from about 4 to 7.5 and suitably dilute concentrations.

The in vitro cytotoxicity of the composition of Example 2 was evaluated using the cell culture system of C3H/10T1/2 C1 8 (10T1/2) mouse embryo fibroblasts. The cells are grown in humidified incubators at 37° C. in an atmosphere of 5% carbon dioxide/air (v/v). 10T1/2 cells are thought to be a spontaneously immortalized, primitive mesenchymal cell line.

The cytotoxicity assays were conducted using standard methods in which 200 cells/60 mm dish were plated and five dishes were prepared for each concentration of Example 2 to be tested. In preliminary screening, it was found that a 1:20 dilution of Example 2 reduced the plating of the cells to 77.8±5.3%. At a 1:2 dilution, the plating efficiency of the cells was reduced to 0% with all cells being killed.

The cytotoxicity was determined to be dose-dependent. It was determined that dilutions in the range of 1:10,000, 1:2,000, 1:1,000 and 1:200 caused little or no cytotoxicity. The plating efficiency of 10T1/2 cells for the following dilutions were determined.

| Dilution | Plating Efficiency % |
|---|---|
| 1:100 | 94.0 |
| 1:50 | 85.4 |
| 1:33.3 | 83.3 |
| 1:25 | 74.0 |
| 1:20 | 67.7 |
| 1:10 | 16.1 |
| 3:20 | 0.0 |

### The cytotoxicity of the composition of Example 2 is therefore dose-dependent in this concentration range.

The LC50 value which reduces the plating efficiency to 50% of that of control cells is estimated to be between a 1:25 and 1:10 dilution of a solution containing 50.0 ug/ml of active ingredients which corresponds with a concentration between 10.0 ug/ml and 25.0 ug/ml. Similarly determined LC50 values for acetaminophen, aspirin and borax are 1,000 ug/ml, 1,500 ug/ml and 2,000 ug/ml.

It should be evident that this disclosure is by way of example and that various changes may be made by adding, modifying or eliminating details without departing from the fair scope of the teaching contained in this disclosure. The invention is therefore not limited to particular details of this disclosure except to the extent that the following claims are necessarily so limited.

What is claimed is:

1. A composition consisting essentially of:
(a) a mixture of lauryl-N-betaine and myristyl-N-betaine, each being present in an amount of from about 0.1 part to about 20 parts,
(b) a cetyl-N,N-dimethylamine oxide being present in an amount of from about 0.1 part to about 20 parts, and
(c) a protonating acid,
the balance being solvent.

2. The composition of claim 1, wherein said lauryl-N-betaine and said myristyl-N-betaine are present in a weight ratio ranging from 30:70 to 70:30.

3. The composition of claim 2, wherein said lauryl-N-betaine and said myristyl-N-betaine are present in a weight ratio ranging from 60:40 to 50:50.

4. The composition of claim 3, wherein said protonating acid is present in an amount sufficient to adjust the pH of the overall composition in the range up from about 4 to about 7.5.

5. The composition of claim 4, wherein said protonating acid is selected from the group consisting of hydrochloric acid, acetic acid and citric acid.

6. The composition of claim 1, wherein said composition provides effective sterilization at temperatures in the range of from about 10° C. to about 45° C.

7. The composition of claim 1, wherein said composition effectively kills Bacillus stearothermophilus.

8. The composition of claim 1, wherein said composition provides effective sterilization of bacteria, fungi, protozoa, virus and spores.

9. The composition of claim 6, wherein said composition provides effective sterilization of microorganisms selected from the group consisting of Pseudomonas aeruginosa, Candida albicans, E. coli, and Bacillus stearothermophilus.

10. The composition of claim 9, wherein said composition is effective against said microorganisms in planktonic cell form, sessile cell form and biofilm form.

11. A method of inhibiting an infection of a microorganism selected from the group consisting of bacteria, fungi, protozoa, virus and spores comprising applying thereto a composition comprising:
(a) a mixture of lauryl-N-betaine and myristyl-N-betaine, each being present in an amount of from about 0.1 part to about 20 parts,
(b) a cetyl-N,N-dimethylamine oxide being present in an amount of from about 0.1 part to about 20 parts, and
(c) a protonating acid,
the balance being solvent.

12. The method of claim 11, wherein said lauryl-N-betaine and said myristyl-N-betaine are present in a weight ratio ranging from 30:70 to 70:30.

13. The method of claim 12, wherein said lauryl-N-betaine and said myristyl-N-betaine are present in a weight ratio ranging from 60:40 to 50:50.

14. The method of claim 13, wherein said protonating acid is present in an amount sufficient to adjust the pH of the overall composition in the range up from about 4 to about 7.5.

15. The method of claim 14, wherein said protonating acid is selected from the group consisting of hydrochloric acid, acetic acid and citric acid.

16. The method of claim 11, wherein said compositions provides effective sterilization at temperatures in the range of from about 10° C. to about 45° C.

17. The method of claim 11, wherein said compositions effectively kills Bacillus stearothermophilus.

18. The method of claim 11, wherein said compositions provides effective sterilization of bacteria, fungi, protozoa, virus and spores.

19. The method of claim 16, wherein said compositions provides effective sterilization of microorganisms selected from the group consisting of Pseudomonas aeruginosa, Candida albicans, E. coli, and Bacillus stearothermophilus.

20. The method of claim 19, wherein said compositions is effective against said microorganisms in planktonic cell form, sessile cell form and biofilm form.

21. A method of disinfecting and lubricating an apparatus having rotational frictionally engaging elements comprising contacting said elements with a composition comprising:
(a) a mixture of lauryl-N-betaine and myristyl-N-betaine, each being present in an amount of from about 0.1 part to about 20 parts,
(b) a cetyl-N,N-dimethylamine oxide being present in an amount of from about 0.1 part to about 20 parts, and
(c) a protonating acid,
rotating said elements, and
contacting said rotating elements with said composition,
the balance being solvent.

22. A method as set forth in claim 21, wherein said apparatus comprises a dental hand piece.

23. A method of sterilizing an apparatus comprising contacting said apparatus with a composition comprising:
(a) a mixture of lauryl-N-betaine and myristyl-N-betaine, each being present in an amount of from about 0.1 part to about 20 parts,
(b) a cetyl-N,N-dimethylamine oxide being present in an amount of from about 0.1 part to about 20 parts, and
(c) a protonating acid,
the balance being solvent,
and maintaining said apparatus in contact with said composition for a time period of about one hour.

24. A method as set forth in claim 23, wherein said betaine and amine oxide components of said composition are present in an amount ranging from about 5% to about 10% by weight based on the total weight of said composition.

25. A method of forming a prophylactic coating on a substrate comprising contacting said substrate with a composition comprising:
(a) a mixture of lauryl-N-betaine and myristyl-N-betaine, each being present in an amount of from about 0.1 part to about 20 parts,
(b) a cetyl-N,N-dimethylamine oxide being present in an amount of from about 0.1 part to about 20 parts, and
(c) a protonating acid,
drying said composition on said substrate to form a substantially continuous film of said composition adhering to said substrate,
the balance being solvent.

26. The method of claim 25, wherein said composition is mixed or agitated to form a foam temporarily sustainable at room temperature and atmospheric pressure, applying said foam to said substrate and collapsing said foam on said substrate to form said substantially continuous film adhering to said substrate.

27. A method of disrupting a biofilm and killing sessile cells forming a matrix of the biofilm comprising applying thereto a composition comprising:
(a) a mixture of lauryl-N-betaine and myristyl-N-betaine, each being present in an amount of from about 0.1 part to about 20 parts,
(b) a cetyl-N,N-dimethylamine oxide being present in an amount of from about 0.1 part to about 20 parts, and
(c) a protonating acid,
the balance being solvent.

28. The method of claim 27, wherein said lauryl-N-betaine and said myristyl-N-betaine are present in a weight ratio ranging from 30:70 to 70:30.

29. The method of claim 28, wherein said lauryl-N-betaine and said myristyl-N-betaine are present in a weight ratio ranging from 60:40 to 50:50.

30. The method of claim 29, wherein said protonating acid is present in an amount sufficient to adjust the pH of the overall composition in the range up from about 4 to about 7.5.

31. The method of claim 27, wherein said composition penetrates, dislodges and/or disintegrates said biofilm.
